(19) United States
(12) Patent Application Publication
(10) Pub. No.: US 2016/0264591 A1
(43) Pub. Date: Sep. 15, 2016

(54) CHIRAL DOPANT AND IDENTIFICATION AND AUTHENTICATION USING POLYMERIC LIQUID CRYSTAL MATERIAL MARKINGS

(71) Applicant: SICPA HOLDING SA, Prilly (CH)

(72) Inventors: Xavier URBANEJA, Savigny (CH); Patrick WYSS, Romont (CH); Sabine MAYADE, Thonon Les Bains (FR); Tristan JAUZEIN, Vevey (CH)

(73) Assignee: SICPA HOLDING SA, Prilly (CH)

(21) Appl. No.: 15/030,668

(22) PCT Filed: Oct. 20, 2014
(86) PCT No.: PCT/EP2014/072402

§ 371 Date: Apr. 20, 2016

Related U.S. Application Data
(60) Provisional application No. 61/893,604, filed on Oct. 21, 2013.

(30) Foreign Application Priority Data
Nov. 8, 2013 (EP) PCT/EP2013/073433

(51) Int. Cl. C07D 493/04; C09K 19/58; G07D 7/12; C09K 19/38; C09D 4/00; C09D 11/03; C09D 11/107

(52) U.S. Cl. CPC C07D 493/04; C09D 11/03; C09K 19/588; C09D 11/107; C09K 19/3809; C09D 4/00; G07D 7/122

(57) ABSTRACT

Disclosed are chiral dopants of general formula (I) below as well as uses thereof involving chiral liquid crystal polymers and markings comprising these polymers: wherein R₁, R₂, R₃, R₄, A₁ and A₂, m, n, o and p are as defined in the claims.

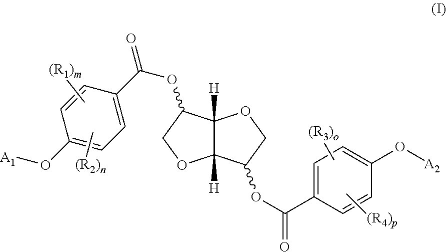

### FIELD OF THE INVENTION

The present invention relates to a new chiral liquid crystal precursor composition useful for the formation of a chiral liquid crystal polymer layer and to new chiral dopants present therein. The invention also relates to a marking comprising a new chiral dopant present therein. The invention also relates to a machine readable marking for the recognition, identification and authentication of individual items. The marking is made of liquid crystal material, which may be applied to a substrate by known variable information printing techniques when referring to digital printing techniques. The marking is made of liquid crystal material, which may be applied also by conventional printing techniques. The marking is detectable and/or identifiable by passive detecting means, e.g. optical filters under non-polarized (ambient) light, as well as by illumination with polarized light. The marking is applied in the form of indicia, such as a one- or two-dimensional bar code, a matrix code, or the like.

### BACKGROUND OF THE INVENTION

“Track and Trace” or authentication systems are currently used in different fields of industry. Many industries suffer from counterfeit or diverted products, in particular in the field of mass-produced items such as beverages, perfumes, pharmaceutical drugs, cigarettes, CDs/DVDs, as well as other kinds of consumables.

Counterfeiting and market diversion are facilitated if the mass products are handled on a lot base, rather than on an individual item base. Counterfeit or diverted products are in such case easily introduced into the supply chain. Producers and retailers would like to be in a position to distinguish their original products from such counterfeit or diverted (parallel imported) products at the level of the individual unit which can be sold.

The underlying technical problem has been addressed in the art through an individual marking of each sellable item introduced into the supply chain. The markings of the prior art were chosen in such a way that they were not susceptible to photocopying, i.e. preferably covert markings were used, which are not visible to the unaided eye or the photocopying machine.

A “Covert” marking, in the context of the present invention, is any marking or security element which cannot be authenticated by the unaided eye, but which, for authentication, depends on a detecting or reading device of any kind, such as an optical filter or an electronic authentication equipment.

An “Overt” marking, in the context of the present invention, is any marking or security element which, for authentication, does not depend on a detecting or reading device; i.e. which can be authenticated by the unaided eye.

“Color” in the context of the present invention is used to designate any spectrally selective return of light (electromagnetic radiation) from an illuminated object, be it in the visible, the infrared or in the UV range of the electromagnetic spectrum (i.e. in the whole wavelength range from 200 nm to 2500 nm).

The term “visible” is used to indicate that a property can be revealed by the unaided eye; “detectable” is used for a property that can be revealed by an dedicated device, though not necessarily by the unaided eye, and “invisible” is used for a property that cannot be detected by the unaided eye. In particular, the term “visible color” means a spectrally selective reflection of light in the wavelength range from 400 nm to 700 nm, which is detectable by the unaided eye.

In the context of the present invention, the terms material and composition are interchangeable.

A first type of individual markings, useful to prevent counterfeit and diversion, is disclosed in U.S. Pat. No. 5,569,317, U.S. Pat. No. 5,502,304, U.S. Pat. No. 5,542,971 and U.S. Pat. No. 5,525,798. According to these documents, a bar code is applied onto the item, using an ink which is not detectable under light of the visible spectrum (400-700 nm wavelength) but which becomes visible when illuminated with light of the UV spectrum (200-380 nm wavelength).

A second type of individual markings is disclosed in U.S. Pat. No. 5,611,958 and U.S. Pat. No. 5,766,324. According to these documents, a marking is applied onto a commercial good, using an ink which is not detectable in the visible spectrum, but which can be detected through illumination with light of the infrared spectrum (800-1600 nm wavelength).

Yet another type of individual markings, applied through an ink, is disclosed in U.S. Pat. No. 5,360,628 and U.S. Pat. No. 6,612,494. This marking needs to be illuminated jointly with UV- and IR-light to be revealed.

Still another type of individual marking relies on inks comprising up-converting phosphors such as described in U.S. Pat. No. 5,698,397.

Most of the markings mentioned in the cited prior art are covert markings, which are completely invisible to the unaided human eye. The reading of such covert markings depends on a corresponding detecting or reading device, able to detect or read the marking. This can be a disadvantage at the retail center or at the point-of-sale, where an appropriate reading device may not always be available.

Overt markings comprising “optically variable” features, e.g. exhibiting viewing-angle dependent color, have been proposed in the art as authentication means for the “person in the street”. Among these are the holograms (cf. Rudolf L. van Renesse, “Optical Document Security” 2^{nd} ed., 1998, chapter 10), the optical thin film security devices (idem, chapter 13) and the liquid crystal security devices (idem, chapter 14).

Particularly useful as security devices are the cholesteric (chiral) liquid crystals. When illuminated with white light, the cholesteric liquid crystal structure reflects light of a determined color, which depends on the material in question and generally varies with the angle of observation when in a cured state and the device temperature when in a non cured state.

U.S. Pat. No. 5,678,863 refers to means for the identification of documents of value which include a paper or polymer region, said region having a transparent and translucent characteristic. A liquid crystal material is applied to the region to produce an optical effect, which differs when viewed in transmitted and reflected light. The liquid crystal material is in liquid form at room temperature and must be enclosed in a containing means such as microcapsules, in order to be used in a printing process such as gravure, roller, spray or ink-jet printing. The printed liquid crystal region can be in the form of a pattern, for example a bar code. The pattern can be verified by visual or machine inspection of the polarization states of the areas having left-handed and right-handed liquid crystal forms.

U.S. Pat. No. 5,798,147 refers to coating compositions of polymerizable liquid crystal monomers which can be applied by conventional printing processes, such as letterpress, rotogravure, flexographic, offset, screen, and ink-jet printing. The printing inks can be used to produce markings and security inscriptions which are invisible to the human eye. The markings can be detected by their circular polarization or their angle dependent reflection color.

U.S. Pat. No. 6,899,824 refers to a process for printing or coating a substrate with a multilayer of a liquid-crystalline composition and at least one non-liquid-crystalline coating. The process and the printed substrate are useful for producing a counterfeit-proof marking of articles. Preferred methods for applying such printing or coating are screen printing, flexographic, and letterpress printing.

Authentication aspects are of crucial importance nowadays, where there is a potential risk of substitution of the original goods by counterfeit or diverted ones. When printing with a chiral liquid crystal polymer precursor composition in the form of a marking such as a data matrix, for example in EP 2285587, a chiral liquid crystal polymer marking finds its place in “Track and Trace” applications. In the field of “Track and Trace” it is desired to combine the marking with at least one security element, able to certify the authenticity of the marked good as an original one.

In the following, “Secure Track and Trace” means the combination of a “Track and Trace” application, which allows for the identification of an individual item, with at least one security element, which additionally allows for the authentication of said item as being genuine.

### Technical Problems

For “Secure Track and Trace” or authentication applications, where a good in open circulation must be marked individually as to its authenticity and identity, and followed throughout its life cycle or for a specific period of time, e.g. for liability reasons, there is a need for a marking which is i) uniquely coded, so as to be identifiable ii) machine-readable, iii) copy (counterfeit) resistant, iv) authenticate-able by the eye of a human user, and v) authenticate-able by a machine.

“Secure Track and Trace” or authentication is often needed in the field of packaging of pharmaceuticals or cigarettes or foodstuffs, with large volumes of items produced every day (frequently more than hundreds of millions and more). Production speed of the items is one of the key points, the production rate being high and the speed of printing being a key component for validating a secure element for a “Secure Track and Trace” or authentication application. There is thus a need for a marking, based on a chiral liquid crystal precursor composition that can be printed quickly and efficiently to provide its color shift effect and secure property.

Another problem is the cost of the chiral dopants in precursor compositions for making a chiral liquid crystal polymer marking. These chiral dopants are often difficult and time-consuming to synthesize, which adversely affects their production costs and thus, the cost of the corresponding markings.

There is therefore a need for a chiral liquid crystal precursor composition which is efficient for use in a high speed line for the production of secure markings attached to an item or to goods, and which can be made at affordable cost.

### SUMMARY OF THE INVENTION

A new chiral dopant family has surprisingly been found which solves the above mentioned problem and can produce, if present in a chiral liquid crystal precursor composition, a secure marking useful for “Secure Track and Trace” or authentication purposes when printed with various printing techniques.

The chiral dopants of the present invention are of general formula (I):

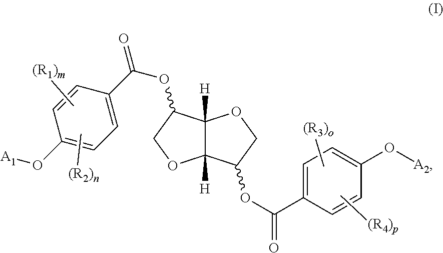

wherein
R₁, R₂, R₃, R₄, R₅, R₆, R₇ and R₈ each independently denote C₁-C₆ alkyl or C₁-C₆ alkoxy;
A₁ and A₂ each independently denote a group:
   (i) —[(CH₂)y-O]z-C(O)—CH═CH₂;
   (ii) —C(O)-D₁-O—[(CH₂)y-O]z-C(O)—CH═CH₂;
   (iii) —C(O)-D₂-O—[(CH₂)y-O]z-C(O)—CH═CH₂;
D₁ denotes a group

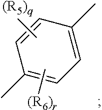

D₂ denotes a group

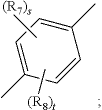

m, n, o, p, q, r, s, and t each independently denote 0, 1 or 2;
y denotes 0, 1, 2, 3, 4, 5 or 6;
z equals 0 if y equals 0 and z equals 1 if y equals 1 to 6.

Embodiments of the chiral dopants of general formula (I) are the chiral dopants of the general formulae (IA) to (ID) below:

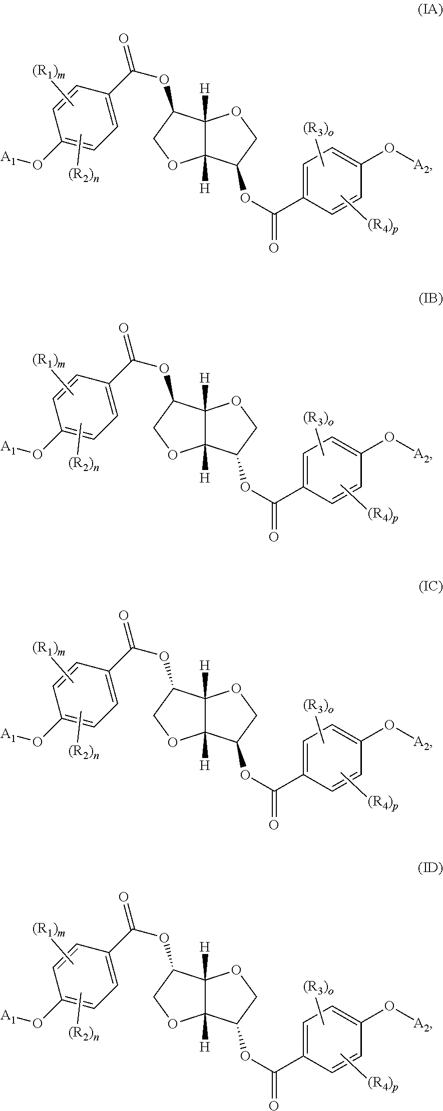

In each of the above formulae (IA), (IB), (IC) and (ID) the meanings of the various symbols are as follows:
R₁, R₂, R₃, R₄, R₅, R₆, R₇ and R₈ each independently denote C₁-C₆ alkyl or C₁-C₆ alkoxy;
A₁ and A₂ each independently denote a group:
   (i) —[(CH₂) y-O]z-C(O)—CH═CH₂;
   (ii) —C(O)-D₁-O—[(CH₂)y-O]z-C(O)—CH═CH₂;
   (iii) —C(O)-D₂-O—[(CH₂)y-O]z-C(O)—CH═CH₂;
D₁ denotes a group

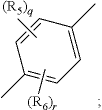

D₂ denotes a group

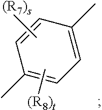

m, n, o, p, q, r, s, and t each independently denote 0, 1 or 2;
y denotes 0, 1, 2, 3, 4, 5 or 6;
z equals 0 if y equals 0 and z equals 1 if y equals 1 to 6.

In the chiral dopant R1, R2, R3, R4, R5, R6, R7 and R8 can each independently denote C1-C6 alkyl.

In the chiral dopant R1, R2, R3, R4, R5, R6, R7 and R8 can each independently denote C1-C6 alkoxy.

In one example, A1 and A2 each independently denote —[(CH2)y-O]z-C(O)—CH═CH2; R1, R2, R3 and R4 each independently denote C1-C6 alkyl or C1-C6 alkoxy; and m, n, o, and p each independently denote 0, 1, or 2.

In another example, A1 and A2 each independently denote —C(O)-D1-O—[(CH2)y-O]z-C(O)—CH═CH2 and/or —C(O)-D2-O—[(CH2)y-O]z-C(O)—CH═CH2; and R1, R2, R3, R4, R5, R6, each independently denote C1-C6 alkyl or C1-C6 alkoxy.

The alkyl or alkoxy groups of R1, R2, R3, R4, R5, R6, R7 and R8 may each independently comprise 1, 2, 3, 4, 5 or 6 carbon atoms.

In accordance with another aspect a chiral liquid crystal precursor composition comprises at least one or more chiral dopants according to the first aspect.

The chiral liquid crystal precursor composition may comprise in addition one or more nematic components.

The chiral liquid crystal precursor composition can contain a security material selected from inorganic luminescent compounds, organic luminescent compounds, IR-absorbers, magnetic materials, forensic markers, and combinations thereof.

The chiral liquid crystal precursor composition may be in a cured chiral liquid crystal state.

A marking for an item or article, wherein said marking comprises chiral polymeric liquid crystal material, which optionally has optical characteristics which allow for its authentication and reading by a machine and/or its authentication by the human eye, wherein the polymer of the chiral polymeric liquid crystal material comprises units derived from one or more chiral dopants according to the first aspect.

The marking of the present invention for the secure tracking or tracing of an item or article comprises a polymeric liquid crystal material comprising units derived from at least one chiral dopant of formula (I) (including chiral dopants of formulae (IA), (IB), (IC) and (ID) set forth above) and having predetermined optical characteristics, which allow for its authentication and reading by a machine, as well as its authentication by the human eye. The marking may be produced on a substrate by a variable information printing process or a conventional printing process in the form of indicia representing a code, which allows for its identification.

The marking may be used for the tracking and/or tracing of an item or article.

Typically, the precursor composition comprises UV-curable reactive monomers or oligomers, and the hardening of the applied composition takes place by UV-curing.

The marking is further preferably designed such that at least a part of it is invisible to the unaided human eye. The marking may be produced on a substrate by a variable information printing process or a conventional printing and could represent a YES/NO information.

In the marking the chiral polymeric liquid crystal material may have a reflection band in the UV spectrum wavelength range of from 200 nm to 400 nm.

In the marking the chiral polymeric liquid crystal material may have a reflection band in the visible spectrum wavelength range of from 400 nm to 700 nm.

In the marking the chiral polymeric liquid crystal material may have a reflection band in the infrared spectrum wavelength range of from 700 to 2500 nm, preferably in the infrared spectrum wavelength range of from 700 nm to 1100 nm.

The chiral polymeric liquid crystal material may have a second reflection band in the wavelength range of from 200 nm to 2500 nm.

The substrate may carry indicia.

The marking of the present invention preferably is applied on items or articles such as value documents, banknotes, passports, identity documents, driving licenses, official permissions, access documents, stamps, tax stamps and banderoles, transportation tickets, event tickets, labels, foils, packaging, spare parts, and consumer goods, which thus carry the marking, either directly—applied to their surface—or indirectly—applied to a label applied to their surface.

The polymeric liquid crystal material of the marking can either be present as a chiral liquid crystal material polymerized on the surface of a substrate, or alternatively consist of pigment flakes of a chiral liquid crystal polymer, comprised in a coating composition applied on a substrate.

Said substrate can be any type of substrate, woven or non-woven, and in particular, it can be made of paper, cardboard, wood, glass, ceramic, metal, plastic, textile, leather, etc.; the substrate may be coated or uncoated, or comprise a sealed or a non-sealed surface.

The chiral polymeric liquid crystal material of the marking of the present invention preferably comprises further security materials, which are present to increase its resistance towards counterfeiting. These security materials are preferably selected from inorganic luminescent compounds, organic luminescent compounds, IR-absorbers, magnetic materials, forensic markers, and combinations thereof.

Said security material can be present as a mere admixture, or, according to the nature of the security material, also as a co-polymerized component of the liquid crystal pigment, of the chiral liquid crystal precursor composition, or of the ink binder. In particular, organic security materials comprising an acrylic or vinylic functionality may easily be co-polymerized into a corresponding main polymer. Alternatively, the security material may be grafted, i.e. chemically linked, onto a pre-existing polymer chain.

The polymeric liquid crystal material may be present in the form of pigment flakes comprised in a suitable binder. For example, suitable binders may include vinylic resins, acrylic resins, styrene-maleic anhydride copolymer resins, polyacetal resins, polyester resins, fatty acid modified polyester resins, and mixtures thereof. The binder may be selected from UV-curable monomers and oligomers of acrylates, vinyl ethers, epoxides and combinations thereof.

The substrate, representing the background on which the chiral liquid crystal material is applied, can be of any color. A white background is a preferred option for realizing a marking which is invisible to the unaided eye in the sense that no visible color is observed. The substrate may in general be selected from reflecting substrates, colored substrates, and transparent substrates.

For enabling an easy authentication by a human user, it is preferred that at least a part of the background on which the liquid crystal material is applied has a contrasting color, such as, for example, red, green, blue or black, which, in combination with the liquid crystal marking, allows to perceive a visible color and an angle-dependent color change by the unaided eye.

The substrate is thus preferably a patterned substrate, comprising at least two differently colored surface areas, each selected from white surface areas, black surface areas, visibly colored surface areas, reflecting surface areas, transparent surface areas, and combinations thereof. It is thus evident to those skilled in the art that the substrate surface carrying the liquid crystal material can have two or more colored areas underneath the chiral liquid crystal material.

The substrate surface on which the chiral liquid crystal material is applied can furthermore carry indicia, which may be of any form or color, such as a pattern, an image, a logo, a text, a 1D- or 2D-barcode or a matrix code, etc. The indicia can be applied by, for example, any printing or coating method.

The substrate can furthermore carry at least one security element selected from inorganic luminescent compounds, organic luminescent compounds, IR-absorbers, magnetic materials, and forensic markers, or combinations thereof. The security element may be present in the form of indicia on the substrate surface or may be incorporated (embedded) in the substrate itself.

The chiral polymeric liquid crystal material is preferably present in the form of indicia, such as a text or a code. Preferred indicia are selected from 1-dimensional, stacked 1-dimensional, and 2-dimensional barcodes. It is also possible that the form of the marking is a cloud of dots where the dots are disposed in such manner that they constitute a binary code or can support optionally encrypted information or is a sum of microglyphs with selective orientation.

The chiral liquid crystal polymer marking of the present invention is preferably produced by applying a chiral liquid crystal precursor composition comprising at least one chiral dopant according to the present invention to a substrate and hardening the composition in the ordered liquid crystal state. The precursor composition usually also comprises reactive monomers or oligomers of at least one nematic liquid crystal compound. The reactive monomers or oligomers are preferably UV-curable; in this case the applied composition is UV-cured and also comprises a photoinitiator system, as known to the skilled person.

The ordered liquid crystal state depends upon the presence of a chiral dopant. Nematic liquid crystals without chiral dopant arrange in a molecular structure which is characterized by birefringence when submitted to a specific temperature very often between 50 to 100° C. Nematic precursors are known from, for example, EP-A-0 216 712, EP-B-0 847 432, and U.S. Pat. No. 6,589,445.

For producing a chiral (i.e. twisted nematic) liquid crystal polymer, the precursor composition must comprise one or more chiral dopants. Said chiral dopants comprise at least one chiral dopant of the above general formula (I) (which includes chiral dopants of any of the above general formulae (IA) to (ID) set forth above) and may comprise additional chiral dopants such as, e.g., the derivatives of isosorbides and isomannides which are disclosed in, e.g., WO 2010/115879.

The chiral dopant induces a helical structure in the nematic liquid crystal compound, characterized by a helical pitch of the order of the wavelength of visible light, leading to light reflection at determined wavelengths, and hence to the appearance of interference color, as well as of angle-dependent color shift. The reflected light from chiral cholesteric liquid crystal phases is circularly polarized (either left-handed or right-handed), according to the rotation sense of the cholesteric helical twist.

The marking of the present invention is obtainable by various techniques, such as those disclosed in, e.g., US2012/0061470 A1 or WO 2010/115879 A2. For example, the marking for an item or article can comprise polymeric liquid crystal material having optical characteristics which allow for its authentication and reading by a machine, as well as its authentication by the human eye. The marking may further be obtainable by independently applying a liquid crystal precursor composition comprising at least one chiral dopant of the above general formula (I) to a substrate, such as by a variable information printing process or conventional printing process, applying heat to both evaporate the solvent contained in the liquid crystal precursor composition and promote the liquid crystal state, and hardening (curing) the applied composition in the ordered liquid crystal state. In case of preparation of flakes for example the step of heating is still needed despite the fact that the composition does not necessarily contain a solvent. The marking may have the form of indicia representing a code, allowing for its identification, other form for identification could also be used instead of an indicia. Thus, in one exemplary embodiment, the liquid crystal precursor composition is first applied to a substrate. The second step involves heating the liquid crystal precursor composition applied onto the substrate to both evaporate the solvent and promote the desired liquid crystal state. The third step involves hardening (curing) the composition on the substrate while it is in the desired liquid crystal state. However, the skilled artisan will appreciate that the above-mentioned sequence may be modified in any way as will be described further below.

The temperature used to evaporate the solvent and to promote the liquid crystal state depends on the liquid crystal monomer composition. According to the present invention, the temperature is chosen preferably between 55° C. and 150° C. and more preferably between 55° C. and 100° C., most preferably between 60° C. and 100° C. The hardening (curing) is preferably performed by subjecting the applied composition to irradiation with UV-light, which induces a polymerization of the reactive monomers or oligomers to form a liquid crystal polymer. The molecular ordering of the liquid crystal is thereby retained, i.e. the cholesteric structure is fixed in the state which was present during the irradiation. In the case of chiral (cholesteric) liquid crystal material, the helical pitch, and herewith the optical properties, such as the reflection color and the angle-dependent color shift remain thus fixed.

Another embodiment of the present invention is a marking according to the invention for an item or article, said marking comprising polymeric liquid crystal material wherein said polymeric liquid crystal material produced on the substrate is obtained by polymerizing a chiral liquid crystal precursor composition comprising at least a chiral dopant according to general formula (I) shown above.

A method for marking an item or article thus comprises the steps of providing a suitable item or article to be marked, and applying at least one chiral polymeric liquid crystal material in the form of indicia representing a code by a variable information printing process or conventional printing process onto the said item or article. In particular, the code represented by the said indicia may be encrypted information, and the method may comprise the step of encrypting the said information.

The said chiral liquid crystal precursor composition can be applied to a substrate by any coating or printing technique. Preferably the composition is applied by a variable information printing process, such as laser printing or ink-jet printing of the continuous or of the drop-on-demand type. Said variable information printing method allows for the unique coding of the marking for each printed item.

For application by ink-jet printing, the composition also comprises an organic solvent in order to adjust the viscosity of the composition to be compatible with the chosen printing process, as known to the skilled person.

For application by continuous ink-jet printing, the composition also comprises a conducting agent (salt) which is soluble in the composition used. Such conducting agent is needed as technical requirement of this printing process, as known to the skilled person.

The chiral liquid crystal precursor composition containing chiral dopant according to formula (I) can also be printed by other printing techniques which do not require any conducting agent in the form of a salt for printing. An example of such a technique is flexographic printing.

The chiral liquid crystal precursor composition according to the invention usually comprises a photoinitiator which is soluble in the composition used. Non-limiting examples of suitable photoinitiators include α-hydroxyketones such as 1-hydroxy-cyclohexyl-phenyl-ketone, a mixture (1:1) of 1-hydroxy-cyclohexyl-phenyl-ketone: benzophenone, 2-hydroxy-2-methyl-1-phenyl-1-propanone or 2-hydroxy-1-[4-(2-hydroxyethoxy)phenyl]-2-methyl-1-propanone, phenylglyoxylate such as methylbenzoylformate or a mixture of oxy-phenyl-acetic acid 2-[2 oxo-2 phenyl-acetoxy-ethoxy]-ethyl ester and oxy-phenyl-acetic 2-[2-hydroxy-ethoxy]-ethyl ester, benzyldimethyl ketal such as alpha, alpha-dimethoxy-alpha-phenylacetophenone, aminoketones such as 2-benzyl-2-(dimethylamino)-1-[4-(4-morpholinyl)phenyl]-1-butanone, 2-methyl-1-[4-(methylthio)phenyl]-2-(4-morpholinyl)-1-propanone, phosphine oxide derivatives such as diphenyl (2,4,6-trimethylbenzoyl)-phosphine oxide or phosphine oxide, phenyl bis(2,4,6-trimethylbenzoyl) supplied by Ciba, and also thioxanthone derivatives such as Speedcure ITX (CAS 142770-42-1), Speedcure DETX (CAS 82799-44-8), Speedcure CPTX (CAS 5495-84-1-2 or CAS 83846-86-0) supplied by Lambson.

The chiral liquid crystal precursor composition according to the invention may also comprise a silane derivative which is soluble in the composition used. Non-limiting examples of silane derivatives that can be used include vinylsilane derivatives such as vinyltriethoxysilane, vinyltrimethoxy silane, vinyltris(2-methoxyethoxy)silane, 3-methacryloxypropyl trimethoxysilane, octyltriethoxysilane and 3-glycidyloxypropyl triethoxysilane from the Dynasylan® family supplied by Evonik.

In general, the method for authenticating an item or article carrying a marking according to the present invention comprises the steps of a) providing an item or article carrying a marking according to the present invention, b) illuminating the marking on said item or article with at least one quality of light from at least one light source, c) detecting the marking's optical characteristics through the sensing of light reflected by the marking, d) determining the item's or article's authenticity from the detected optical characteristics of the marking.

The marking comprises a cholesteric liquid crystal material, exhibiting spectrally selective, viewing angle-dependent light reflection, the reflected light having a particular sense of circular polarization.

The light source may be a spectrally selective light source.

The light source may be selected from ambient light, incandescent light, laser diodes, light emitting diodes, and these light sources having color filters.

The light source may emit in a spectral domain selected from one or more of the visible (400-700 nm wavelength), the near infrared (700-1100 nm wavelength), the far infrared (1100-2500 nm wavelength) and the UV (200-400 nm wavelength) region of the electromagnetic spectrum.

The illuminating may be performed by a light source selected from unpolarized, linear polarized, left circular polarized, and right circular polarized light sources.

At least two different light sources may be used in conjunction.

The detecting may be performed using at least one optical filter selected from linear polarizing, left circular polarizing, right circular polarizing filters and the electro-optic polarization switches. The optical filter may be combined with a color filter. At least two different optical filters may be used in conjunction.

The detecting may be performed by the human eye. Alternatively or in addition, the detecting may be performed by electro-optic detection equipment. The electro-optic detecting equipment may be selected from photocells, linear CCD image sensor arrays, 2-dimensional CCD image sensor arrays, linear CMOS image sensor arrays, and 2-dimensional CMOS image sensor arrays.

The marking of the present invention can be authenticated according to a first method by simple visual inspection under ambient light. To this aim, the background, on which the liquid crystal material is applied, must provide sufficient optical contrast, so as to allow the human observer to perceive the reflected color and the color shift of the liquid crystal material. Depending on the background, part of the marking may remain virtually invisible to the unaided eye.

In a second method, the marking is authenticated under ambient light with the help of a passive detecting means such as an optical filter. A preferred corresponding passive detecting means is a left-handed or a right-handed circular polarizing filter, or a juxtaposition of both. This allows determining the rotation sense of the helical pitch of the chiral liquid crystal material by determining the polarization state of the light reflected by the material. Optionally, the polarization filter can be combined with color filters, in order to reduce the spectral bandwidth to the spectral reflection band of the liquid crystal material, and hence to reduce background contributions. More than one optical filter may be used in conjunction.

In a third method, the marking is authenticated with the help of circular polarized light from at least one polarized light source. The liquid crystal material reflects differently light of different circular polarization; hence materials of left and of right helical pitch can be distinguished by their respective response to circularly polarized light. The illumination of the marking by the polarized light source, as well as the observation of the reflected light from the marking, may optionally be performed through a color filter. More than one polarized light source may be used in conjunction.

In a fourth method, the marking is authenticated with the help of an electro-optical authentication device. In a first embodiment, said device comprises at least one photocell, in combination with a circular polarization filter and/or with a circular polarized light source. In another embodiment said electro-optical device comprises an camera, such as a linear CCD sensor array, a 2-dimensional CCD image sensor array, a linear CMOS image sensor array, or a 2-dimensional CMOS image sensor array, in combination with a circular polarization filter and/or with a circular polarized light source.

Optionally, the circular polarization filter or the circular polarized light source in the above embodiments can be combined with color filters, to select a particular spectral domain and to enhance the contrast ratio of the light reflected from the liquid crystal material to the light reflected from the background.

The circular polarization filters can generally also be replaced by an electro-optical polarization switch. Such a device is known in the art, e.g. from DE 102 11 310 B4, and allows to select one or the other circular polarization state by an applied corresponding voltage.

The (chiral) marking of the present invention may be authenticated by verifying one or more of its characteristic properties, such as the circular polarization state and/or the viewing-angle dependent color of the reflected light from the marking. The polarized light source or the polarized light detection equipment or both may be chosen to operate in the visible, the infrared, or the UV region of the electromagnetic spectrum, or in a combination of these, according to the optical characteristics of the marking.

The marking of the present invention can be identified by reading the indicia it represents and by subsequently correlating the information so retrieved from the marking with information stored in a database. In a particular embodiment, the information represented by the indicia of the marking is encrypted, and said identification comprises the step of decrypting said information. Preferably, the indicia are read by an electro-optic camera, such as an electro-optic sensor array, for example, a linear of two-dimensional CCD- or a CMOS-image sensor array.

In general, the method for identifying an item or article carrying a marking according to the present invention comprises the steps of a) providing an item or article carrying a marking according to the present invention, b) illuminating the marking on said item or article with at least one quality of light from at least one light source, c) reading the indicia represented by the marking, deriving corresponding information, d) correlating the information retrieved from the indicia of the marking with information stored in a data base, e) obtaining confirmation or denial concerning the item's or articles identity. Alternatively the method for identifying an item or article carrying a marking according to the present invention comprises the steps of a) providing an item or article carrying a marking according to the present invention, b) illuminating the marking on said item or article with at least one quality of light from at least one light source, c) reading the indicia represented by the marking, deriving corresponding information, d) correlating the information retrieved from the indicia of the marking with information not stored in a data base, e) obtaining confirmation or denial concerning the item's or articles identity.

The identification of an item or article carrying a marking according to the present invention can be performed with the same reading equipment configuration or assembly which is used for the authentication.

In a first embodiment the said indicia are represented by a 1-dimensional or a 2-dimensional barcode, and the image retrieved by the electro-optic camera in digital form is analyzed using a corresponding algorithm. The information contained in the barcode is retrieved, if necessary, decrypted, and compared with information stored in a database, thereby identifying the item, and optionally updating the database with supplementary information, e.g. about the item's history. The camera may be part of a reading device equipped with own communication capabilities, or part of a communication device, such as a mobile phone, the retrieval of the information taking place using the mobile phone's internal resources. The database may be either located in the communication device (built-in or exchangeable memory), or on an external server reached via a communication network.

In a second embodiment the said indicia are represented by an alphanumeric code, and the image retrieved by the electro-optic camera (reading device) in digital form is analyzed using a corresponding optical character recognition (OCR) algorithm. The information contained in the code is retrieved and compared with information stored in a database, thereby identifying the item, and optionally updating the database. As in the first embodiment, the database may be either located in the reading device (built-in or exchangeable memory), or on an external server reached via a communication network. The alphanumeric code can be printed using a standard font or a special machine identifiable font. Alternatively, the alphanumeric code can be visually read and either sent via a communication system (e.g. Internet or SMS) to a data center for validation or checked against data provided with the item in form of a label, reference mark or another alphanumeric code.

The marking of the present invention, made of chiral polymeric liquid crystal material having determined optical characteristics, can be used for the secure tracking and tracing of items, articles or goods with an individualized, counterfeit-resistant code for the secure tracking and tracing of said items, articles or goods.

The application of an individualized code onto a good or item requires a variable information printing process. A preferred variable information printing process in the context of the present invention is selected from continuous ink-jet printing and drop-on-demand ink-jet printing; these printing processes allow for a rapid, non-contact application of said individualized code onto any kind of surfaces. Said individualized code allows the identification of each single item at a later stage of its life cycle.

In order to prevent the substitution of the original item by a counterfeit carrying a copy of said individualized code, said individualized code must be counterfeit-resistant. Counterfeit resistance can be provided through a particular security material having particular physical, preferably optical, properties; said material can be either constitutive or incorporated in the marking. The particular security material can be a chiral polymeric liquid crystal material having determined optical characteristics, or in addition with the chiral polymeric liquid crystal material an additive selected from inorganic luminescent compounds, organic luminescent compounds, IR-absorbers, magnetic materials, forensic markers, and combinations thereof.

The marking of the present invention can be used on items or articles such as, for example, value documents, banknotes, passports, identity documents, driving licenses, official permissions, access documents, stamps, tax stamps and banderoles (in particular for tobacco products and alcoholic beverages), transportation tickets, event tickets, labels, foils, packaging (in particular for pharmaceutical products), and in general for marking spare parts and consumer goods (in particular to address liability issues).

The marking of the present invention, applied to items, goods, or articles is suitable for use in the secure tracking and tracing of such marked items, goods or articles. Such secure tracking and tracing of an item or article comprises in particular, the first commutable steps of a) applying a marking according to the invention to the item or article to be traced; and b) storing information related to the marked item or article in a data base; as well as the second commutable steps of c) authenticating the item or article according to the authentication method disclosed herein; and d) identifying the item or article, according to the identification method disclosed herein, using the information previously stored in the database. Optionally the database may thereby be updated with new information elements related to the item or article.

The code applied to the item or good represents digital information, which is stored in a database, in order to identify the item or good at a later stage. Said code may be encrypted, so as to protect the information it contains upon transmission from and to the database. Said database can be part of a database management system. All kind of encryption algorithms are suitable, e.g. a public-private key of the RSA type.

Said database may be a local database, integrated into the authentication device. Alternatively, it may be a remote database, linked to the authentication device through a wired or wireless connection. A local database may also be updated regularly from a remote server.

Preferably, the information exchange with the database takes place in encrypted form.

In a further aspect, the present invention provides the application of the individual marking by a variable-information printing process. Preferred is ink-jet printing, either using the continuous ink-jet or the drop-on-demand (DOD) ink-jet or valve-jet printing process. Industrial ink-jet printers, commonly used for numbering and coding applications on conditioning lines and printing presses, are particularly suitable. Preferred ink-jet printers are single nozzle continuous ink-jet printers (also called raster or multi level deflected printers) and drop-on-demand ink-jet, in particular valve-jet, printers. In a further aspect, the present invention provides the application of the individual marking by a non variable-information printing process. Preferred is analogic printing process such as flexographic techniques.

There is also provided a method for the secure tracking or tracing of an item or article, wherein the marking is used and which comprises the first commutable steps of
a. applying said marking to the item or article;
b. storing information related to the marked item or article in a database;
   and the second commutable steps of
c. authenticating the item or article;
d. identifying the item or article, using the information stored in the database;
   and the optional step of
e. updating the database with new information elements related to the item or article.

Another aspect provides an intermediate in the liquid crystal state, obtainable by independently applying a liquid crystal precursor composition comprising the chiral dopant to a substrate by a variable information printing process or a conventional printing process and applying heat to both evaporate the solvent contained in the liquid crystal precursor composition and promote the liquid crystal state.

Usually to provide for an entirely covert and machine readable marking, nematic liquid crystal materials are used. With the chiral dopant according to the present invention we can provide for an overt or semi-covert and covert machine readable marking, cholesteric or chiral-nematic liquid crystal materials.

The chiral dopants of the above general formula (I) (including embodiments thereof represented by the above general formulae (IA), (IB), (IC) and (ID)) used in the chiral liquid crystal precursor composition can also be employed to provide other types of markings.

For example, additional embodiments of the present invention include the following:

(A) A liquid crystal polymer marking which is obtainable by a process comprising:
(i) applying a chiral liquid crystal precursor composition comprising one or more chiral dopants of general formula (I) set forth above onto a substrate;
(ii) heating the applied composition to bring same to a first chiral liquid crystal state;
(iii) applying to one or more areas of the applied composition at least one of
   (1) at least one modifying composition which modifies the first chiral liquid crystal state locally in the one or more areas, and
   (2) at least one modifying composition which upon heating modifies the first chiral liquid crystal state locally in the one or more areas;
(iv) in the case of (2), heating at least one of the one or more areas to bring same to at least one of a second chiral liquid crystal state and an isotropic state; and
(v) curing and/or polymerizing the thus locally modified precursor composition to convert same into a liquid crystal polymer marking.

In one aspect of the above marking (A), the at least one modifying composition may at least partially convert the first chiral liquid crystal state into a (predominantly or substantially) isotropic state. In another aspect, the at least one modifying composition may at least partially convert the first chiral liquid crystal state into a second chiral liquid crystal state that is different from the first state. In yet another aspect, in stage (iii) at least two or at least three different modifying compositions may be applied simultaneously or consecutively.

In another aspect of the marking (A), the chiral liquid crystal precursor composition may comprise (i) one or more (e.g. two, three, four, five or more and in particular, at least two) different nematic compounds and (ii) one or more (e.g., two, three, four, five or more) different chiral dopant compounds which include at least one chiral dopant of general formula (I) according to the present invention and are capable of giving rise to a cholesteric state of the chiral liquid crystal precursor composition upon heating. Further, the one or more nematic compounds may comprise at least one compound which comprises at least one polymerizable group. For example, all of the one or more nematic compounds and all of the one or more chiral dopant compounds which include at least one chiral dopant of general formula (I) according to the present invention may comprise at least one polymerizable group. The at least one polymerizable group may, for example, comprise a group which is able to take part in a free radical polymerization and in particular, a (preferably activated) unsaturated carbon-carbon bond such as, e.g., a group of formula H₂C═CH—C(O)—.

In a further aspect of the marking (A), the at least one modifying composition may comprise at least one compound that is selected from ketones having from 3 to about 6 carbon atoms (e.g., 3, 4, 5 or 6 carbon atoms), alkyl esters and dialkylamides of carboxylic acids which comprise a total of from 2 to about 6 carbon atoms (e.g., 2, 3, 4, 5 or 6 carbon atoms), dialkyl sulfoxides comprising a total of from 2 to about 4 carbon atoms (e.g., 2, 3 or 4 carbon atoms), and optionally substituted nitrobenzene. For example, the at least one modifying agent may comprise at least one of dimethyl ketone, methyl ethyl ketone, ethyl acetate, dimethyl formamide, dimethyl sulfoxide, and nitrobenzene.

In another aspect of the above marking (A), the at least one modifying composition may comprise a second chiral liquid crystal precursor composition. The first chiral liquid crystal precursor composition and the second chiral liquid crystal precursor composition may be identical. Alternatively, the first and second chiral liquid crystal precursor compositions may be different from each other. For example, the second chiral liquid crystal precursor composition may differ from the first chiral liquid crystal precursor composition at least in that the second composition comprises at least one of the one or more chiral dopant compounds which include at least one chiral dopant of general formula (I) according to the present invention in a concentration which is different from a concentration of the same chiral dopant compound in the first composition and/or at least in that the second composition comprises at least one chiral dopant compound which include at least one chiral dopant of general formula (I) according to the present invention which is different from any of the one of the one or more chiral dopant compounds which include at least one chiral dopant of general formula (I) according to the present invention that are present in the first composition.

In yet another aspect of the marking (A), the at least one modifying composition may comprise a chiral dopant composition. The chiral dopant composition may comprise, for example, at least one chiral dopant compound which is a dopant of general formula (I) according to the present invention. In another aspect, the chiral dopant composition may further (or instead) comprise at least one chiral dopant compound which is different from a chiral dopant of formula (I).

In another aspect of the marking (A), the modifying composition may further comprise at least one resin and/or at least one salt and/or at least one pigment and/or dye that absorbs in the visible or invisible region of the electromagnetic spectrum and/or at least one luminescent pigment and/or dye.

In another aspect of the marking (A), stage (ii) of the process may comprise a heating of the applied composition to a temperature of from about 55° C. to about 150° C., e.g., from about 55° C. to about 100° C., or from about 60° C. to about 100° C.

In a still further aspect of the marking (A), stage (iii) of the process may comprise an application (e.g., deposition) of the at least one modifying composition by continuous ink-jet printing and/or drop-on-demand ink-jet printing and/or spray printing and/or valve-jet printing.

In another aspect, immediately after stage (iii) of the process a stream of air may be passed over the surface of the one or more areas, preferably (substantially) parallel thereto.

In yet another aspect, the marking (A) may be in the form of at least one of an image, a picture, a logo, indicia, or a pattern representing a code selected from one or more of 1-dimensional barcodes, stacked 1-dimensional barcodes, 2-dimensional barcodes, 3-dimensional barcodes, and a data matrix, a cloud of dots where the dots are disposed in such manner that they constitute a binary code or can support optionally encrypted information, a sum of microglyphs with specific orientation.

The present invention also provides a substrate which comprises (e.g., carries on a surface thereof) the marking (A) as set forth above, including the various aspects thereof. In one aspect of the substrate, the marking may serve as at least one of a security element an identification element, and a tracking and tracing element. In another aspect, the substrate may be, or comprise, at least one of an identity document, a label, packaging, a banknote, a security document, a passport, a stamp, an ink-transfer film, and a reflective film, a capsule, a pill, a cork, a spare part, a watch, a timepiece.

The present invention also provides an ink that comprises (i) one or more nematic compounds and (ii) one or more chiral dopants of general formula (I) above. These re typically capable of giving rise to a cholesteric state of the ink upon application of heat thereto.

Typically, the ink is a security ink.

There is also provided a flake or coating that comprises (i) one or more nematic compounds and (ii) one or more chiral dopants of general formula (I) above.

The present invention also provides a method of providing a substrate with a liquid crystal polymer marking (A). The method comprises:
(i) applying a first chiral liquid crystal precursor composition comprising at least one chiral dopant of general formula (I) set forth above (including chiral dopants of general formulae (IA), (IB), (IC) and (ID) set forth above) onto at least one surface of a (solid) substrate;
(ii) heating the applied composition to bring same to a first chiral liquid crystal state;
(iii) applying to one or more areas of the applied composition at least one of:
   (1) at least one modifying composition which modifies the first chiral liquid crystal state locally in the one or more areas, and
   (2) at least one modifying composition which upon heating modifies the first chiral liquid crystal state locally in the one or more areas;
(iv) in the case of (2), heating at least one of the one or more areas to bring same to at least one of a second chiral liquid crystal state and an isotropic state; and
(v) at least one of curing and polymerizing the thus locally modified precursor composition to convert same into a liquid crystal polymer marking.

The present invention also provides a substrate which comprises a marking (e.g., on at least one (outer) surface thereof). The marking comprises a layer or film of a chiral liquid crystal polymer made from a chiral liquid crystal precursor composition comprising at least one chiral dopant of general formula (I) set forth above. The layer or film comprises in at least one area (region) thereof a liquid crystal polymer that has at least one optical property which is different from an optical property of the remainder of the layer or film. In one aspect of the substrate, the liquid crystal polymer in the at least one area of the layer or film may comprise (e.g., may be in) an isotropic state. In another aspect, the liquid crystal polymer in the at least one area of the layer or film may comprise (e.g., may be in) an isotropic state.

The present invention also provides a substrate which comprises a marking (e.g., on at least one (outer) surface thereof). The marking comprises a layer or film of a first chiral liquid crystal polymer made from a chiral liquid crystal precursor composition comprising at least one chiral dopant of general formula (I) set forth above that has a first optical property. The layer or film comprises in at least one area (region) thereof a second liquid crystal polymer that has at least one second optical property which is different from the first optical property.

The present invention also provides a substrate which comprises a marking (e.g., on at least one (outer) surface thereof). The marking comprises a layer or film of a chiral liquid crystal polymer made from a chiral liquid crystal precursor composition comprising at least one chiral dopant of formula (I) set forth above in a first chiral liquid crystal state. The layer or film comprises in at least one area (region) thereof a liquid crystal polymer in a second chiral liquid crystal state that has at least one optical property which is different from an optical property of the polymer in the first chiral liquid crystal state.

For further information regarding the liquid crystal polymer marking (A) and the related subject matter set forth above, US 2011/0135889 A1, US 2011/0135890 A1, US 2011/0133445 A1 and US 2011/0135853 A1 may, for example, be referred to.

(B) A chiral liquid crystal precursor composition comprising one or more chiral dopants of general formula (I) set forth above and at least one salt that changes a position of a selective reflection band exhibited by the composition in a cured state compared to a position of a selective reflection band exhibited by a composition in a cured state that does not contain the at least one salt, the chiral liquid crystal precursor composition.

In one aspect, the chiral liquid crystal precursor composition (B) may comprise (i) one or more (e.g. two, three, four, five or more and in particular, at least two) different nematic compounds A and (ii) one or more (e.g., two, three, four, five or more) different chiral dopant compounds which comprise at least one chiral dopant of general formula (I) set forth above and are capable of giving rise to a cholesteric state of the chiral liquid crystal precursor composition upon heating. Further, the one or more nematic compounds A may comprise at least one compound which comprises at least one polymerizable group. For example, all of the one or more nematic compounds and all of the one or more chiral dopant compounds may comprise at least one polymerizable group. The at least one polymerizable group may, for example, comprise a group which is able to take part in a free radical polymerization and in particular, a (preferably activated) unsaturated carbon-carbon bond such as, e.g., a group of formula H₂C═CH—C(O)—.

In another aspect of the precursor composition (B) the at least one salt (e.g., one, two, three or more different salts) that changes the position of the selective reflection band exhibited by the cured chiral liquid crystal precursor composition may be selected from metal salts and (preferably quaternary) ammonium salts. For example, the at least one salt may comprise at least one salt of a metal such as an alkali or alkaline earth metal (e.g., Li, Na), for example, one or more of lithium perchlorate, lithium nitrate, lithium tetrafluoroborate, lithium bromide, lithium chloride, sodium carbonate, sodium chloride, sodium nitrate, and/or one or more (organically substituted) ammonium salts such as tetraalkylammonium salts, for example, one or more of tetrabutylammonium perchlorate, tetrabutylammonium chloride, tetrabutylammonium tetrafluoroborate, and tetrabutylammonium bromide.

In another aspect of precursor composition (B), the at least one salt may shift the position of the selective reflection band exhibited by the cured chiral liquid crystal precursor composition by at least 5 nm, e.g., by at least 10 nm, by at least 20 nm, by at least 30 nm, by at least 40 nm, or by at least 50 nm, and/or the at least one salt may shift the position of the selective reflection band to shorter wavelengths or may shift the position of the selective reflection band to longer wavelengths and/or the shifted position of the selective reflection band may be in the visible range and/or the shifted position of the selective reflection band may be in the invisible range. In this regard, it is noted that “shifting the position of the selective reflection band” as used herein and in the appended claims means shifting λₘₐₓ as measured using an analytical spectral device that measures the reflectance of a sample in the infrared or near-infrared-or visible or UV range of the spectrum, such as the LabSpec Pro device made by Analytical Spectral Devices Inc. of Boulder, Colo.

In yet another aspect of the precursor composition (B), the at least one salt may be present in the precursor composition in a concentration of at least 0.01%, e.g., at least 0.05%, at least 0.1%, or at least 0.5% by weight, based on the solids content (i.e., without volatile components such as solvents) of the precursor composition. In another aspect, the at least one salt may be present in a concentration of not higher than %, e.g., not higher than 5%, or not higher than 2% by weight, based on the solids content of the precursor composition.

In another aspect, the precursor composition (B) may be present in the form of at least one of an image, a picture, a logo, indicia, and a pattern representing a code selected from one or more of a 1-dimensional barcode, a stacked 1-dimensional barcode, a 2-dimensional barcode, a 3-dimensional barcode, and a data matrix, a cloud of dots where the dots are disposed in such manner that they constitute a binary code or can support optionally encrypted information, a sum of microglyphs with specific orientation representing a code.

The present invention further provides the precursor composition (B) set forth above (including the various aspects thereof) in a cured (chiral liquid crystal) state.

The present invention further provides a substrate which comprises at least one precursor composition (B) as set forth above (including the various aspects thereof), either in an uncured state or in a cured (chiral liquid crystal) state. In one aspect of the substrate, the substrate may be or comprise at least one of a label, packaging, a cartridge, a container, a closed cartridge (e.g., a capsule) that contains pharmaceuticals, nutraceuticals, foodstuffs or a beverage (such as, e.g., coffee, tea, milk, chocolate, etc.), a banknote, a credit card, a stamp, a tax label, a security document, a passport, an identity card, a driver's license, an access card, a transportation ticket, an event ticket, a voucher, an ink-transfer film, a reflective film, an aluminum foil, and a commercial good. The substrate may further be a film or sheet of polyethylene terephthalate (PET) or polyolefin such as polyethylene, for example as a temporary support from which the cured precursor composition (e.g., in the form of a marking) can be transferred to a permanent substrate (e.g., one of the substrates set forth in the preceding sentence).

The present invention further provides a method of changing the position of a selective reflection band exhibited by a chiral liquid crystal precursor composition comprising at least one chiral dopant of general formula (I) set forth above in the cured (chiral liquid crystal) state. The method comprises incorporating in the precursor composition at least one salt that is capable of changing the position of the selective reflection band exhibited by the precursor composition in a cured (chiral liquid crystal) state.

The present invention further provides a method of shifting a selective reflection band exhibited by a chiral liquid crystal precursor composition comprising at least one chiral dopant of general formula (I) set forth above in the cured chiral liquid crystal state to a predetermined position. The method comprises incorporating in the precursor composition at least one salt in an amount that results in a shift of the selective reflection band to the predetermined position.

In one aspect, the method may further comprise a determination (e.g., recording) of the shift of the position of the selective reflection band as a function of the amount (concentration) of the least one salt that is present in the precursor composition (e.g., by plotting the shift of the position of the selective reflection band against the amount of salt) and the selection of the amount of the at least one salt that results in the shift of the selective reflection band of the cured precursor composition to the (desired) predetermined position. In another aspect of the method, the position of the selective reflection band may be shifted to a predetermined wavelength.

The present invention further provides a method of providing a marking on a substrate. The method comprises (a) the application of at least one chiral liquid crystal precursor composition (B) as set forth above (including the various aspects thereof) onto a surface of a substrate (one or more areas). The method further comprises (b) the heating of the applied chiral liquid crystal precursor composition to bring same to a chiral liquid crystal state; and (c) the curing of the composition in the chiral liquid crystal state (e.g., by radiation, such as UV-radiation).

In one aspect of the method, the chiral liquid crystal precursor composition (B) may be heated to a temperature of from about 55° C. to about 150° C. to bring the chiral liquid crystal precursor composition to a chiral liquid crystal state. In another aspect of the method, the liquid crystal precursor composition may be applied onto the surface of the substrate by at least one of spray printing, knife coating, roller coating, screen coating, curtain coating, gravure printing, flexography, screen-printing, pad printing, and ink-jet printing (for example continuous ink-jet printing, drop-on-demand ink-jet printing, valve-jet printing), and/or may be applied in the form of at least one of an image, a picture, a logo, indicia, and a pattern representing a code selected from one or more of a 1-dimensional barcode, a stacked 1-dimensional barcode, a 2-dimensional barcode, a 3-dimensional barcode, and a data matrix, a cloud of dots where the dots are disposed in such manner that they constitute a binary code or can support optionally encrypted information, a sum of microglyphs with specific orientation representing a code.

In a still further aspect, the substrate may be or may comprise at least one of a label, packaging, a cartridge, a container, a closed cartridge (e.g., a capsule) that contains pharmaceuticals, nutraceuticals, foodstuffs or a beverage (such as, e.g., coffee, tea, milk, chocolate, etc.), a banknote, a credit card, a stamp, a tax label, a security document, a passport, an identity card, a driver's license, an access card, a transportation ticket, an event ticket, a voucher, an ink-transfer film, a reflective film, an aluminum foil, and a commercial good.

In another aspect of the method, at least two different precursor compositions (e.g., 2, 3, 4 or more different compositions), at least one of them being a precursor composition according to the present invention may be applied (either separately or simultaneously) onto the substrate and may be processed according to (a) and (b) either together or separately. For example, the precursor compositions may differ with respect to the at least one salt comprised therein and/or they may differ with respect to the concentration of the at least one salt (same salt) comprised therein.

The present invention further provides a substrate that is provided with a marking, wherein the substrate is obtainable by the method set forth above (including the various aspects thereof).

For further information regarding the chiral liquid crystal precursor composition (B) and the related subject matter set forth above, WO 2012/076533 may, for example, be referred to.

(C) A substrate having thereon a marking or layer comprising a cured chiral liquid crystal precursor composition comprising one or more chiral dopants of general formula (I) set forth above and at least one salt that changes a position of a selective reflection band exhibited by the cured composition compared to a position of a selective reflection band exhibited by the cured composition that does not contain the at least one salt. A modifying resin made from one or more polymerizable monomers is disposed between the substrate and the marking or layer and in contact with the marking or layer in one or more areas thereof, the modifying resin changing a position of the selective reflection band exhibited by the cured chiral liquid crystal precursor composition comprising the at least one salt on the substrate in the one or more areas.

The present invention also provides a marking or layer that is locally modified by a modifying resin as such (i.e., without the presence of a substrate). The term “modifying resin” as used in the present specification and in the appended claims includes cured resins as set forth below, and also includes aqueous resins such as, e.g., polyamide resins (for example, CAS No 175893-71-7, CAS No 303013-12-9, CAS No 393802-62-5, CAS No 122380-38-5, CAS No 9003-39-8), alkyd resins (e.g. of the polyester type), and polyacrylates.

In one aspect of the substrate/marking or layer (C), the chiral liquid crystal precursor composition may comprise (i) one or more (e.g. two, three, four, five or more and in particular, at least two) different nematic compounds A and (ii) one or more (e.g., two, three, four, five or more) different chiral dopant compounds B which comprise at least one chiral dopant of general formula (I) set forth above and are capable of giving rise to a cholesteric state of the chiral liquid crystal precursor composition upon heating. Further, the one or more nematic compounds A may comprise at least one compound which comprises at least one polymerizable group. For example, all of the one or more nematic compounds A and all of the one or more chiral dopant compounds B may comprise at least one polymerizable group. The at least one polymerizable group may, for example, comprise a group which is able to take part in a free radical polymerization and in particular, a (preferably activated) unsaturated carbon-carbon bond such as, e.g., a group of formula H₂C═CH—C(O)—.

In another aspect of the substrate/marking or layer (C) the at least one salt (e.g., one, two, three or more different salts) that changes the position of the selective reflection band exhibited by the cured chiral liquid crystal precursor composition (regardless of whether or not the polymer is present on a substrate) may be selected from metal salts and (preferably quaternary) ammonium salts. For example, the at least one salt may comprise at least one salt of a metal such an alkali or alkaline earth metal (e.g., Li, Na), for example, one or more of lithium perchlorate, lithium nitrate, lithium tetrafluoroborate, lithium bromide, lithium chloride, sodium carbonate, sodium chloride, sodium nitrate, and/or one or more (organically substituted) ammonium salts such as tetraalkylammonium salts, for example, one or more of tetrabutylammonium perchlorate, tetrabutylammonium chloride, tetrabutylammonium tetrafluoroborate, and tetrabutylammonium bromide.

In a further aspect of the substrate/marking or layer (C) at least one of the one or more polymerizable monomers for providing the modifying resin for changing the position of the selective reflection band exhibited by the cured chiral liquid crystal precursor composition may comprise at least two unsaturated carbon-carbon bonds and/or at least one of the one or more polymerizable monomers may comprise at least one heteroatom, preferably selected from O, N and S and in particular, O and/or N. For example, at least one of the one or more polymerizable monomers for providing the modifying resin may comprise one or more groups (e.g., one, two, three, four, five, six, or more groups) of formula H₂C═CH—C(O)— or H₂C═C(CH₃)—C(O)—. Non-limiting examples of corresponding monomers include polyether acrylates, modified polyether acrylates (such as, e.g., amine-modified polyether acrylates), polyester acrylates, modified polyester acrylates (such as, e.g., amine-modified polyester acrylates), hexafunctional polyester acrylates, tetrafunctional polyester acrylates, aromatic difunctional urethane acrylates, aliphatic difunctional urethane acrylates, aliphatic trifunctional urethane acrylates, aliphatic hexafunctional urethane acrylates, urethane monoacrylates, aliphatic diacrylates, bisphenol A epoxy acrylates, modified bisphenol A epoxy acrylates, epoxy acrylates, modified epoxy acrylates (such as, e.g., fatty acid modified epoxy acrylates), acrylic oligomers, hydrocarbon acrylate oligomers, ethoxylated phenol acrylates, polyethylene glycol diacrylates, propoxylated neopentyl glycol diacrylates, diacrylated bisphenol A derivatives, dipropylene glycol diacrylates, hexanediol diacrylates, tripropylene glycol diacrylates, polyether tetraacrylates, ditrimethylol propane tetraacrylates, dipentaerythritol hexaacrylates, mixtures of pentaerythritol tri- and tetraacrylates, dipropylene glycol diacrylates, hexanediol diacrylates, ethoxylated trimethylol propane triacrylates, and tripropylene glycol diacrylates.

In another aspect of the substrate/marking or layer (C), the modifying resin for changing the position of the selective reflection band exhibited by the (salt-containing) cured chiral liquid crystal precursor composition may comprise a radiation-cured resin, for example, a UV-cured resin. Another type of resin that can be used in the present invention are aqueous resins, such as polyamide resins, for example CAS No 175893-71-7, CAS No 303013-12-9, CAS No 393802-62-5, CAS No 122380-38-5, CAS No 9003-39-8.

In another aspect of the substrate/marking or layer (C), the modifying resin may shift the position of the selective reflection band exhibited by the salt-containing cured chiral liquid crystal precursor composition by at least 5 nm and/or may shift the position to shorter wavelengths and/or the shifted position of the selective reflection band may be in the visible range. In this regard, it is noted that “shifting the position of the selective reflection band” as used herein and in the appended claims means shifting λₘₐₓ as measured using an analytical spectral device that measures the reflectance of a sample in the infrared-near-infrared-visible-UV range of the spectrum, such as the LabSpec Pro device made by Analytical Spectral Devices Inc. of Boulder, Colo.

In yet another aspect of the substrate/marking or layer (C), at least one of the one or more areas of the substrate which carry the modifying resin may be in the form of at least one of an image, a picture, a logo, indicia, and a pattern representing a code selected from one or more of a 1-dimensional barcode, a stacked 1-dimensional barcode, a 2-dimensional barcode, a 3-dimensional barcode, and a data matrix, and/or at least a part of the cured chiral liquid crystal precursor composition may be in the form of at least one of an image, a picture, a logo, indicia, and a pattern representing a code selected from one or more of a 1-dimensional barcode, a stacked 1-dimensional barcode, a 2-dimensional barcode, a 3-dimensional barcode, and a data matrix, a cloud of dots where the dots are disposed in such manner that they constitute a binary code or can support optionally encrypted information, a sum of microglyphs with specific orientation representing a code.

In a still further aspect, the substrate (C) may be or comprise at least one of a label, packaging, a cartridge, a container or a capsule that contains pharmaceuticals, nutraceuticals, foodstuffs or a beverage (such as, e.g., coffee, tea, milk, chocolate, etc.), a banknote, a credit card, a stamp, a tax label, a security document, a passport, an identity card, a driver's license, an access card, a transportation ticket, an event ticket, a voucher, an ink-transfer film, a reflective film, an aluminum foil, and a commercial good. The marking according to the present invention can also be created on a substrate such as, e.g., a film or sheet of polyethylene terephthalate (PET) or polyolefin such as polyethylene for later transfer to a permanent substrate (e.g., one of the substrates set forth in the preceding sentence).

The present invention further provides a method of providing a marking on a substrate. The method comprises the application of a curable chiral liquid crystal precursor composition onto a surface of a substrate which carries in one or more areas of the surface of the substrate a modifying resin made from one or more polymerizable monomers. The curable chiral liquid crystal precursor composition comprises at least one chiral dopant of general formula (I) set forth above and at least one salt which changes the position of the selective reflection band exhibited by the cured chiral liquid crystal precursor composition (when in a chiral liquid crystal state) compared to the position of the selective reflection band exhibited by the cured chiral liquid crystal precursor composition that does not contain the at least one salt. Further, the modifying resin is capable of changing the position of the selective reflection band exhibited by the salt-containing cured chiral liquid crystal precursor composition on the substrate in the one or more areas in which it is present. The curable chiral liquid crystal precursor composition is applied in such a way that the composition covers at least a part of the one or more areas that carry the modifying resin and also covers at least one area of the surface of the substrate that does not carry the modifying resin. The method further comprises the heating of the applied chiral liquid crystal precursor composition to bring same to a chiral liquid crystal state; and the curing of the composition in the chiral liquid crystal state (e.g., by radiation, such as UV-radiation).

In one aspect of the method, the chiral liquid crystal precursor composition may be heated to a temperature of from about 55° C. to about 150° C. to bring the chiral liquid crystal precursor composition to a chiral liquid crystal state. In another aspect of the method, the liquid crystal precursor composition may be applied onto the substrate by at least one of spray printing, knife coating, roller coating, screen coating, curtain coating, gravure printing, flexography, screen-printing, pad printing, and ink-jet printing (for example continuous ink-jet printing, drop-on-demand ink-jet printing, valve-jet printing), and/or may be applied in the form of at least one of an image, a picture, a logo, indicia, and a pattern representing a code selected from one or more of a 1-dimensional barcode, a stacked 1-dimensional barcode, a 2-dimensional barcode, a 3-dimensional barcode, and a data matrix, a cloud of dots where the dots are disposed in such manner that they constitute a binary code or can support optionally encrypted information, a sum of microglyphs with specific orientation representing a code.

In yet another aspect of the method, the modifying resin may be present in at least one of the one or more areas in the form of at least one of an image, a picture, a logo, indicia, and a pattern representing a code selected from one or more of a 1-dimensional barcode, a stacked 1-dimensional barcode, a 2-dimensional barcode, a 3-dimensional barcode, and a data matrix and/or may have been provided on the substrate by at least one of continuous ink-jet printing, drop-on-demand ink-jet printing, valve-jet printing, spray printing, flexography, gravure printing, offset, dry offset printing, letterpress printing, pad printing and screen printing.

In a still further aspect of the method, the substrate may be or may comprise at least one of a label, packaging, a cartridge, a container or a capsule that contains pharmaceuticals, nutraceuticals, foodstuffs or a beverage (such as, e.g., coffee, tea, milk, chocolate, etc.), a banknote, a credit card, a stamp, a tax label, a security document, a passport, an identity card, a driver's license, an access card, a transportation ticket, an event ticket, a voucher, an ink-transfer film, a reflective film, an aluminum foil, and a commercial good.

In another aspect, the modifying resin may be capable of shifting the position of the selective reflection band exhibited by the (salt-containing) cured chiral liquid crystal precursor composition on the substrate by at least 5 nm.

The present invention further provides a substrate that is provided with a marking and is obtainable by the method of the set forth above (including the various aspects thereof).

The present invention also provides a method of shifting the position of the selective reflection band exhibited by a chiral liquid crystal precursor made from a composition comprising (i) one or more nematic compounds, (ii) one or more chiral dopant compounds which comprise at least one chiral dopant of general formula (I) set forth above and are capable of giving rise to a cholesteric state of the chiral liquid crystal precursor, and (iii) at least one salt that changes the position of the selective reflection band exhibited by the cured composition compared to the position of the selective reflection band exhibited by the cured composition that does not contain the at least one salt. The method comprises contacting the chiral liquid crystal precursor composition with a modifying resin which is made from one or more polymerizable monomers, at least one of the monomers comprising a heteroatom selected from O, N, and S, and is capable of changing the position of the selective reflection band exhibited by the cured chiral liquid crystal precursor composition. The chiral liquid crystal precursor composition is then heated to a temperature of from about 55° C. to about 150° C. to bring it to a chiral liquid crystal state. Thereafter the chiral liquid crystal precursor composition is cured.

In one aspect of the method, the position of the selective reflection band may be shifted by at least about 5 nm. Other aspects of the method such as, e.g., aspects relating to components (i), (ii) and (iii) include those set forth above with respect to the substrate/marking or layer (C).

For further information regarding the substrate/marking or layer (C) and the related subject matter set forth above, WO 2012/076534 may, for example, be referred to.

(D) A marking on an item or substrate, the marking comprising a layer or pattern of a chiral liquid crystal polymer composition that exhibits an initial set of optical properties and is made by curing a chiral liquid crystal precursor composition that comprises one or more chiral dopants of general formula (I) set forth above in a chiral liquid crystal state. The layer or pattern comprises
(1) one or more first areas exhibiting a first modified set of optical properties that is different from the initial set of optical properties and is obtainable by contacting the precursor composition in the one or more first areas with a first modifying agent;
(2) one or more second areas exhibiting a second modified set of optical properties that is different from the initial set of optical properties and different from the first modified set of optical properties and is obtainable by contacting the precursor composition in the one or more second areas with a second modifying agent that is of a different type than the first modifying agent.

In one aspect of the marking (D), at least one of the one or more first areas may be partially or completely overlapped by at least one second area and/or at least one of the one or more second areas may be partially or completely overlapped by at least one first area. In another aspect, at least one of the one or more first areas may not be overlapped by any second area and/or at least one of the one or more second areas may not be overlapped by any first area.

In yet another aspect of the marking (D), the initial and the first and second modified sets of optical properties may differ with respect to at least one property of light that is reflected by the chiral liquid crystal polymer composition. For example, the at least one property may be selected from one or more of the spectrum, the polarization, and λₘₐₓ of the reflected light.

In a still further aspect of the marking (D), the initial and the first and second modified sets of optical properties may comprise at least one property that is indicative of an optically anisotropic state of the chiral liquid crystal polymer composition.

In another aspect of the marking (D), the chiral liquid crystal precursor composition may comprise (i) one or more nematic compounds, (ii) one or more chiral dopant compounds B that are capable of giving rise to a cholesteric state of the chiral liquid crystal precursor composition and comprise at least one chiral dopant of general formula (I) set forth above, and (iii) at least one salt that changes a maximum wavelength of the selective reflection band (λₘₐₓ) exhibited by the polymer composition compared to a maximum wavelength of the selective reflection band (λₘₐₓ) exhibited by a polymer composition that does not contain the at least one salt.

In one aspect, the one or more nematic compounds may comprise at least one compound that comprises at least one polymerizable group. The at least one polymerizable group may comprise, for example, an unsaturated carbon-carbon bond such as a group of formula H₂C═CH—C(O)—. In another aspect, the one or more nematic compounds and all of the one or more chiral dopant compounds B may comprise at least one polymerizable group.

In another aspect, the at least one salt may be selected from metal salts and ammonium salts. For example, the at least one salt may comprise at least one of lithium perchlorate, lithium nitrate, lithium tetrafluoroborate, lithium bromide, lithium chloride, tetrabutylammonium perchlorate, tetrabutylammonium chloride, tetrabutylammonium tetrafluoroborate, tetrabutylammonium bromide, sodium carbonate, sodium chloride, and sodium nitrate.

In another aspect of the marking (D), the precursor composition may be in an initial optically anisotropic state and in the one or more first areas the initial optically anisotropic state may be changed to a first modified optically anisotropic state, and in the one or more second areas the initial optically anisotropic state may be changed to a second modified optically anisotropic state or converted to an optically isotropic state. In another aspect, the precursor composition may be in an initial chiral liquid crystal state and in the one or more first areas the initial chiral liquid crystal state may be changed to a first modified chiral liquid crystal state by the first modifying agent, and in the one or more second areas the initial chiral liquid crystal state may be changed to a second modified chiral liquid crystal state or may be changed to a non-chiral liquid crystal state by the second modifying agent.

In yet another aspect of the marking (D), the first modifying agent may be solid and/or semisolid and the second modifying agent may be fluid and/or the first modifying agent may be virtually unable to penetrate the precursor composition and the second modifying agent may at least partially penetrate the precursor composition.

In another aspect, the first modifying agent may be or may comprise a resin that is made from one or more polymerizable monomers. Further, at least one of the one or more polymerizable monomers may comprise at least two unsaturated carbon-carbon bonds and/or may comprise at least one heteroatom selected from O, N and S. Merely by way of example, at least one of the one or more polymerizable monomers may comprise at least one group of formula H₂C═CH—C(O)— or H₂C═C(CH₃)—C(O)—. In another aspect, the resin may comprise a radiation-cured resin such as a UV-cured resin and/or the resin may comprise a dried aqueous resin.

In another aspect, the second modifying agent may be fluid and selected from one or more of (a) a modifying composition that comprises at least one compound selected from ketones having from 3 to about 6 carbon atoms, alkyl esters and dialkylamides of carboxylic acids which comprise a total of from 2 to about 6 carbon atoms, dialkyl sulfoxides comprising a total of from to about 4 carbon atoms, and optionally substituted nitrobenzene, (b) a modifying composition that comprises at least one chiral liquid crystal precursor composition, and (c) a modifying composition that comprises at least one chiral dopant composition.

In yet another aspect of the marking (D), the first modifying agent may be selected from a solid or semisolid cured and/or dried resin made from one or more polymerizable monomers, and both the first modifying agent and the second modifying agent may change an initial maximum wavelength of the selective reflection band (λₘₐₓ) exhibited by the precursor composition in the chiral liquid crystal state.

In a still further aspect, the first modifying agent and the second modifying agent may act from opposite sides of the layer or pattern of the precursor composition. For example, the first modifying agent may be arranged between the substrate and the layer or pattern in the one or more first areas and the second modifying agent may act from the side opposite the substrate in the one or more second areas.

In another aspect of the marking (D), the one or more first areas and/or the one or more second areas may be in the form of at least one of an image, a picture, a logo, indicia, and a pattern representing a code selected from one or more of a 1-dimensional barcode, a stacked 1-dimensional barcode, a 2-dimensional barcode, a 3-dimensional barcode, a cloud of dots where the dots are disposed in such manner that they constitute a binary code or can support optionally encrypted information, and a data matrix and/or at least a part of the layer or pattern may be in the form of at least one of an image, a picture, a logo, indicia, and a pattern representing a code selected from one or more of a 1-dimensional barcode, a stacked 1-dimensional barcode, a 2-dimensional barcode, a 3-dimensional barcode, a cloud of dots where the dots are disposed in such manner that they constitute a binary code or can support optionally encrypted information, a sum of microglyphs with specific orientation representing a code and a data matrix.

In another aspect, the item or substrate may be or may comprise at least one of a label, packaging, a cartridge, a container or a capsule that contains foodstuffs, nutraceuticals, pharmaceuticals, or beverages, a banknote, a credit card, a stamp, a tax label, a security document, a passport, an identity card, a driver's license, an access card, a transportation ticket, an event ticket, a voucher, an ink-transfer film, a reflective film, an aluminum foil, and a commercial good.

The present invention also provides a method of providing a marking on an item or substrate as well as an item or substrate produced by this method. The method comprises:
a) applying onto a surface of an item or substrate which carries a first modifying agent in one or more first areas a curable chiral liquid crystal precursor composition which comprises at least one chiral dopant of general formula (I) set forth above and assumes an initial chiral liquid crystal state upon heating it in such a way that the composition covers at least a part of the one or more first areas, the first modifying agent being able to modify the initial chiral liquid crystal state of the composition;
b) heating the applied composition to bring the same to a first modified chiral liquid crystal state in the one or more first areas and to the initial chiral liquid crystal state in all other areas, if any, of the applied composition;
c) applying to one or more second areas of the applied composition at least one second modifying agent which is of a different type than the first modifying agent and (1) is able to locally modify the initial and/or first modified chiral liquid crystal states provided by b), or (2) is able to locally modify the initial and/or first modified chiral liquid crystal states provided by b) upon heating the composition;
d) in the case of (2), heating the composition at least in the one or more second areas; and
e) curing/polymerizing the entire thus modified precursor composition to produce a liquid crystal polymer marking on the item or substrate.

In one aspect of the method, at least one of the one or more first areas may be partially or completely overlapped by at least one second area and/or at least one of the one or more second areas may be partially or completely overlapped by at least one first area. In another aspect of the method, at least one of the one or more first areas may not be overlapped by any second area and/or at least one of the one or more second areas may not be overlapped by any first area.

In yet another aspect of the method, the initial and first and second modified sets of optical properties may differ with respect to at least one property of light that is reflected by the chiral liquid crystal polymer composition. For example, the at least one property may be selected from one or more of the spectrum, the polarization, and λₘₐₓ of the reflected light.

In a still further aspect of the method, the initial and first and second modified sets of optical properties may comprise at least one property that is indicative of an optically anisotropic state of the chiral liquid crystal polymer composition and/or is indicative of a conversion of an optically anisotropic state to an optically isotropic state of the composition.

In another aspect, the chiral liquid crystal precursor composition may comprise (i) one or more nematic compounds A, (ii) one or more chiral dopant compounds B which comprise at least one chiral dopant of general formula (I) set forth above and are capable of giving rise to a cholesteric state of the chiral liquid crystal precursor composition, and (iii) at least one salt that changes a maximum wavelength of the selective reflection band (λₘₐₓ) exhibited by the polymer composition compared to a maximum wavelength of the selective reflection band (λₘₐₓ) exhibited by a polymer composition that does not contain the at least one salt.

In a still further aspect of the method, step b) and/or step d) may comprise a heating of the precursor composition to a temperature of from about 55° C. to about 150° C.

In another aspect of the method, the precursor composition may be applied by at least one of spray printing, knife coating, roller coating, screen coating, curtain coating, gravure printing, flexography, screen-printing, pad printing, continuous ink-jet printing, drop-on-demand ink-jet printing, and valve-jet printing and/or the precursor composition may be applied in the form of at least one of a (continuous or discontinuous) layer, an image, a picture, a logo, indicia, and a pattern representing a code selected from one or more of a 1-dimensional barcode, a stacked 1-dimensional barcode, a 2-dimensional barcode, a 3-dimensional barcode, a cloud of dots where the dots are disposed in such manner that they constitute a binary code or can support optionally encrypted information, a sum of microglyphs with specific orientation representing a code and a data matrix.

In another aspect of the method, the first modifying agent may have been provided on the item or substrate in the one or more first areas by at least one of spray printing, knife coating, roller coating, screen coating, curtain coating, gravure printing, flexography, offset printing, dry offset printing, letterpress printing, screen-printing, pad printing, continuous ink-jet printing, drop-on-demand ink-jet printing, and valve-jet printing and/or the first modifying agent may be present on the item or substrate in the one or more first areas in the form of at least one of an image, a picture, a logo, indicia, and a pattern representing a code selected from one or more of a 1-dimensional barcode, a stacked 1-dimensional barcode, a 2-dimensional barcode, a 3-dimensional barcode, a cloud of dots where the dots are disposed in such manner that they constitute a binary code or can support optionally encrypted information, a sum of microglyphs with specific orientation representing a code and a data matrix.

In yet another aspect, the second modifying agent may be applied in the one or more second areas by at least one of continuous ink-jet printing, drop-on-demand ink-jet printing, spray printing, and valve-jet printing and/or the second modifying agent may be applied in the one or more second areas in the form of at least one of an image, a picture, a logo, indicia, and a pattern representing a code selected from one or more of a 1-dimensional barcode, a stacked 1-dimensional barcode, a 2-dimensional barcode, a 3-dimensional barcode, a cloud of dots where the dots are disposed in such manner that they constitute a binary code or can support optionally encrypted information, a sum of microglyphs with specific orientation representing a code and a data matrix.

In a still further aspect, the item or substrate may be or may comprise at least one of a label, packaging, a cartridge, a container or a capsule that contains foodstuffs, beverages, nutraceuticals or pharmaceuticals, a banknote, a credit card, a stamp, a tax label, a security document, a passport, an identity card, a driver's license, an access card, a transportation ticket, an event ticket, a voucher, an ink-transfer film, a reflective film, an aluminum foil, and a commercial good.

For further information regarding the marking (D) and the related subject matter set forth above, WO 2012/163778 may, for example, be referred to.

A coding flake or film (E) comprising
(i) at least two chiral liquid crystal polymer (CLCP) layers comprising a first CLCP layer that has a first detectable parameter and a second CLCP layer including a second detectable parameter, at least one of the first and second CLCP layers made from a CLCP precursor composition comprising one or more chiral dopants of general formula (I) set forth above;
(ii) at least one additional layer including a third detectable parameter, the at least one additional layer comprising a material that is not a chiral liquid crystal polymer;
   at least the third detectable parameter being different from each of the first detectable parameter and the second detectable parameter.

In one aspect of the coding flake or film (E), each of the first detectable parameter, the second detectable parameter and the third detectable parameter may be different so that the coding flake or film includes at least three different detectable parameters.

In another aspect of the coding flake or film (E), the at least one additional layer may be positioned between the first CLCP layer and the second CLCP layer.

In another aspect of the coding flake or film (E),the first detectable parameter and the second detectable parameter may comprise circular reflected polarized light. For example, the first detectable parameter and the second detectable parameter may comprise a difference between reflected wavelengths of at least 10 nm, e.g., at least 20 nm or at least 30 nm and/or the difference between reflected wavelengths may be in a range of from 20 nm to 80 nm.

In another aspect of the coding flake or film (E), the at least one additional layer may include a material selected from at least one of magnetic material, absorber material absorbing electromagnetic radiation in at least one of the UV, visible and IR range, luminescent material, photochromic material, and thermochromic material and/or the additional layer may include an opaque material and/or a colored material.

In yet another aspect of the coding flake or film (E), the at least two CLCP layers may include the same color shift properties, or they may include different color shift properties.

In a still further aspect of the coding flake or film (E), the at least two CLCP layers may comprise the same chiral liquid crystal precursor composition and/or the at least two CLCP layers may be formulated to have a difference in pitch.

In another aspect of the coding flake or film (E), the at least two CLCP layers may comprise different chiral liquid crystal precursor compositions wherein at least one or the at least two CLCP layers contains a chiral dopant according to formula (I) or (IA) and/or (IB) and/or (IC) and/or (ID) In another aspect of the coding flake or film (E), the at least two CLCP layers may comprise different chiral liquid crystal precursor compositions.

In another aspect of the coding flake or film (E), the first detectable parameter and the second parameter may comprise at least one property selected from circular reflected polarized light, position of at least one spectral reflection band, visibility with unaided eye, and thickness of layer.

In further aspects of the coding flake or film (E), at least two chiral liquid crystal polymer (CLCP) layers may comprise a reflection band in the visible range of the electromagnetic spectrum and/or each of the at least two CLCP layers may comprise a reflection band in the invisible range of the electromagnetic spectrum and/or the at least two CLCP layers include at least one layer comprising a reflection band in the visible range of the electromagnetic spectrum and at least one layer comprising a reflection band in the invisible range of the electromagnetic spectrum.

In another aspect of the coding flake or film (E), the first detectable parameter may comprise a first optically measurable parameter, the second detectable parameter may comprise a second optically measurable parameter, and the third detectable parameter may comprise a third optically or magnetically measurable parameter.

In another aspect of the coding flake or film (E), the at least one additional layer may comprise a magnetic material, for example, at least one material selected from ferromagnetic materials, ferrimagnetic materials, paramagnetic materials, and diamagnetic materials and/or at least one material selected from metals and metal alloys comprising at least one of iron, cobalt, nickel, and gadolinium. In particular, the magnetic material may comprise at least one material selected from inorganic oxide compounds, ferrites of formula MFe₂O₄ wherein M represents Mg, Mn, Co, Fe, Ni, Cu or Zn, and garnets of formula A₃B₅O₁₂ wherein A represents La, Ce, Pr, Nd, Sm, Eu, Gd, Tb, Dy, Ho, Er, Tm, Yb, Lu or Bi and B represents Fe, Al, Ga, Ti, V, Cr, Mn or Co. In another aspect, the magnetic material may comprise at least one of a soft magnetic material and a hard magnetic material.

In another aspect of the coding flake or film (E), the at least one additional layer may comprise a luminescent material comprising one or more lanthanide compounds such as, for example, at least one complex of a lanthanide and a β-diketo compound.

In another aspect of the coding flake or film (E), the at least one additional layer may comprise at least one magnetic material and at least one lanthanide compound.

In another aspect of the coding flake or film (E), the flake or film may consist of the first CLCP layer, the second CLCP layer and the additional layer so that only three layers are present, or the flake or film may comprise at least two additional layers arranged between the first CLCP layer and the second CLCP layer. For example, each of the at least two additional layers may comprise at least one detectable parameter. The at least one detectable parameter of each of the at least two additional layers may include at least one detectable parameter that is different.

In another aspect of the coding flake or film (E), the flake or film has a total thickness of from 5 μm to 100 μm and/or each CLCP layer may have a thickness of from 2 μm to 3 μm, and the additional layer may have a thickness of 1 μm or greater.

In another aspect of the coding flake or film (E), the at least two CLCP layers each may have a thickness of from 2 μm to 30 μm.

The present invention also provides an ink or coating composition which comprises at least one type of coding flake (E).

In one aspect, the ink or coating composition may comprise from 0.01% to 30% by weight, preferably from 0.01% to 20% by weight, more preferably from 0.1% to 3% by weight, even more preferably from 0.2% to 1% by weight of the flakes (E), based on a total weight of the ink or coating composition. In another aspect, the at least one type of coding flake may comprise a plurality of different types of coding flakes. In yet another aspect, the coding flakes may have at least two different sizes and/or the average diameter of the flakes may be between 3 to 30 times the total layer thickness.

The present invention also provides a marking on an article of value or an item which comprises a plurality of coding flakes (E) and/or an ink or coating composition as set forth above.

In one aspect of the marking, the coding flakes may be randomly distributed and/or may comprise different types of coding flakes and/or may have at least two different sizes.

In another aspect, the article or item may comprise at least one of a label, packaging, a cartridge, a container or capsule that contains foodstuffs, beverages, nutraceuticals or pharmaceuticals, a banknote, a credit card, a thread, a stamp, a tax label, an anti-tamper seal, a security document, a passport, an identity card, a driver's license, an access card, a transportation ticket, an event ticket, a voucher, an ink-transfer film, a reflective film, an aluminum foil, a commercial good a capsule, a cork, and a lottery ticket.

In yet another aspect, the above marking may comprise at least one of a barcode, a data matrix, and a stripe, a logo, a solid print, and a cloud of dots where the dots are disposed in such manner that they constitute a binary code or can support optionally encrypted information visible or invisible to the unaided eyes and/or the flakes may be at least one of overprinted, down-printed, and coated above or below a barcode, data matrix or stripe.

In a still further aspect of the above marking, the flake density may be not higher than 1000 flakes/mm², preferably not higher than 100 flakes/mm², more preferably not higher than 35 flakes/mm², even more preferably not higher than 7 flakes/mm².

The present invention also provides a method of marking a substrate, article of value or item, wherein the method comprises:
providing the substrate, article or item with a marking comprising a plurality of coding flakes (E) as set forth above (including the various aspects thereof);
reading at least one of deterministic data and non-deterministic data of the marking; and
recording and storing in a computer database the deterministic and/or non-deterministic data representative of the marking.

The present invention also provides a method of identifying and/or authenticating a substrate, article of value or item, wherein the method comprises:
reading at least one of deterministic data and non-deterministic data of a marking associated with the substrate, article or item, the marking including a plurality of coding flakes (E) as set forth above (including the various aspects thereof); and
comparing using a database through a computer the read data with stored data of the deterministic and/or non-deterministic data of the plurality of coding flakes in the marking.

In aspects of the above methods, reading may be performed with a reading device comprising at least illumination elements and optical detection elements and/or the reading device may further include magnetic detection and/or the plurality of flakes may comprise the same coding flakes or the plurality of flakes may comprise different coding flakes.

In other aspects of the above methods, the non-deterministic data may comprise the distribution of the flakes of the plurality of flakes within the marking and/or the non-deterministic data may comprise the size of the flakes within the marking and/or the deterministic data may comprise at least one of magnetism, absorption, reflectance, fluorescence, luminescence, particle size and polarization. For example, the non-deterministic data may comprise the distribution of flakes of the plurality of flakes within the marking and the deterministic data may include magnetism.

In still other aspects of the above methods, the deterministic data may further include at least one optical property and/or the coding flakes may be randomly distributed and/or the flakes may be provided on the substrate, article or item by at least one of printing, coating or bronzing with a liquid, semi-solid or solid composition that comprises at least one type of flakes (E).

The present invention also provides a method of marking an article or item, which method comprises providing the article or item with at least one marking that comprises a plurality of coding flakes (E) as set forth above (including the various aspects thereof).

The present invention also provides a marking comprising a random distribution of coding flakes (E) as set forth above (including the various aspects thereof) wherein the random distribution is detectable in an area of at least 1 mm².

In one aspect of the marking, the random distribution may be detectable in an area of at least 100 mm² and/or the random distribution may comprise from 3 to 1000 flakes, for example, from 30 to 100 flakes.

The present invention also provides an item including an identification and/or authentication mark, which item comprises in at least one area thereof randomly distributed coding flakes (E) as set forth above (including the various aspects thereof) at a flake density of not higher than 100 flakes per square millimeter, for example, 30 to 100 flakes per square millimeter.

The present invention also provides a coating composition for marking and identifying an item, which coating composition comprises coding flakes (E) as set forth above (including the various aspects thereof) at a concentration of from 0.01% to 20% by weight, e.g., at a concentration of from 0.2% and 1% by weight.

The present invention also provides a mixture of flakes comprising a plurality of coding flakes (E) as set forth above (including the various aspects thereof).

In one aspect, the mixture of flakes may include flakes having at least one of the first detectable parameter, the second detectable parameter and the third detectable parameter that is different from other flakes in the mixture of flakes and/or the at least one of the first detectable parameter, the second detectable parameter and the third detectable parameter may include at least one of reflectance, fluorescence, luminescence, flake size, magnetic property, polarization and absorption.

The present invention also provides film which is used to obtain coding flakes (E) as set forth above (including the various aspects thereof).

The present invention also provides a security document or an item including a coding flake (E) as set forth above (including the various aspects thereof) and/or a mixture of flakes as set forth above, wherein the mixture of flakes comprises a combination of randomly distributed flakes in the form of a marking that has a maximum area of 9 to 100 mm².

The present invention also provides a method of marking a security document or an item comprising associating a mixture of flakes as set forth above with the security document or an item so that at least one of the first detectable parameter, the second detectable parameter and the third detectable parameter is a categorizing parameter. For example, the third detectable parameter may be the categorizing parameter.

For further information regarding coding flake or film (E) and the related subject matter set forth above, US 2013/0256415 A1 may, for example, be referred to.

The present invention also provides a tamper proof structure, a secure laminate structure, a randomly distributed marking a security feature comprising at least in one of its component or composition a chiral dopant according to formula (I) and/or formula I(A) and/or formula I(B) and/or formula I(C) and/or formula I(D).

The present invention also provides the use of a chiral dopant according to formula (I) and/or formula I(A) and/or formula I(B) and/or formula I(C) and/or formula I(D) in a tamper proof structure, a secure laminate structure, a randomly distributed marking a security feature comprising at least in one of its component or composition.

### DESCRIPTION OF THE DRAWINGS

For a more complete understanding of the present invention reference is made to the detailed description of the invention and the attached drawings.

FIG. 1 schematically depicts a cardboard packaging, e.g. a pharmaceutical packaging, carrying representative markings a), b), c) according to the invention, which are printed with chiral liquid crystal material at different locations on said cardboard packaging:

a) shows a Data Matrix code on a particularly dark colored background, e.g. a black background;

b) shows a Data Matrix code on a background of mixed color, e.g. having dark and light colored parts;

c) shows a Data Matrix code on a white background.

FIG. 2 shows images captured from an ECC200 data matrix code printed with UV-cured liquid crystal (LC) material on coated cardboard:

a) LC Data Matrix code retrieved from a black background, under right-circular polarized white light illumination, having a right-circular polarizing filter in front of the CMOS camera.

b) LC Data Matrix code retrieved from a black/white background, under right-circular polarized white light illumination, having a right-circular polarizing filter in front of the CMOS camera.

c) LC Data Matrix code retrieved from a black/white background, under right-circular polarized white light illumination, having no filter in front of the CMOS camera.

d) LC Data Matrix code retrieved from a black/white background, under unpolarized white light illumination, having no filter in front of the CMOS camera.

FIG. 3a is a diagram which shows the wavelength of the maximum normal reflection (λₘₐₓ) of a chiral polymeric liquid crystal material as a function of the concentration of a comparative chiral dopant in the dry composition.

FIG. 3b is a diagram which shows the wavelength of the maximum normal reflection (λₘₐₓ) of a chiral polymeric liquid crystal material as a function of the concentration of a comparative chiral dopant of the prior art in the dry composition (as shown in FIG. 3a) and additionally, as a function of the concentration of a chiral dopant of formula (I) according to the present invention in the dry composition.

### DETAILED DESCRIPTION OF THE INVENTION

The present invention provides new chiral dopants of formula (I):

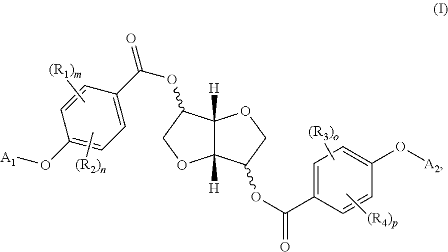

wherein
R₁, R₂, R₃, R₄, R₅, R₆, R₇ and R₈ each independently denote C₁-C₆ alkyl or C₁-C₆ alkoxy;
A₁ and A₂ each independently denote a group:
   (i) —[(CH₂)y-O]z-C(O)—CH═CH₂;
   (ii) —C(O)-D₁-O—[(CH₂)y-O]z-C(O)—CH═CH₂;
   (iii) —C(O)-D₂-O—[(CH₂)y-O]z-C(O)—CH═CH₂;
D₁ denotes a group

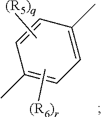

D₂ denotes a group

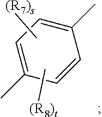

m, n, o, p, q, r, s, and t each independently denote 0, 1 or 2;
y denotes 0, 1, 2, 3, 4, 5 or 6;
z equals 0 if y equals 0 and z equals 1 if y equals 1 to 6.

Embodiments of the chiral dopants of formula (I) are the chiral dopants of the general formulae (IA) to (ID):

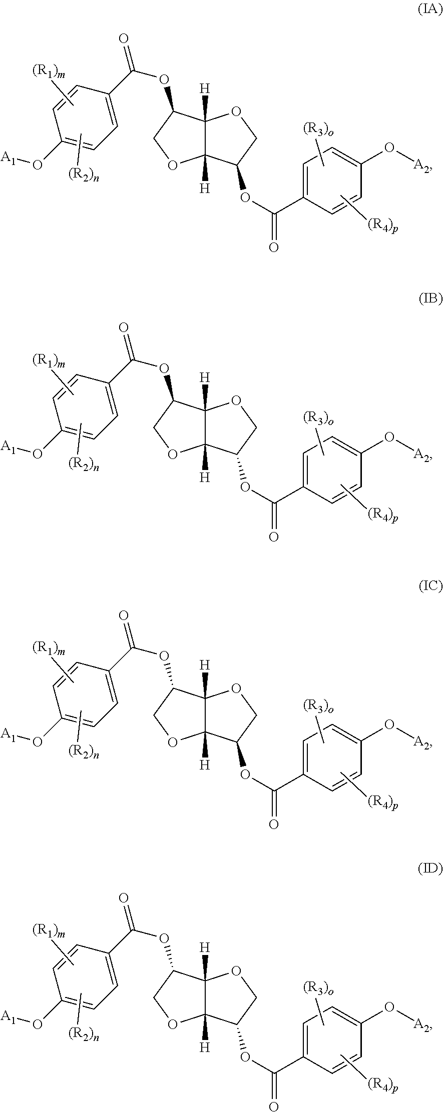

wherein
R₁, R₂, R₃, R₄, R₅, R₆, R₇ and R₈ each independently denote C₁-C₆ alkyl or C₁-C₆ alkoxy;
A₁ and A₂ each independently denote a group:
   (i) —[(CH₂)y-O]z-C(O)—CH═CH₂;
   (ii) —C(O)-D₁-O—[(CH₂)y-O]z-C(O)—CH═CH₂;
   (iii) —C(O)-D₂-O—[(CH₂)y-O]z-C(O)—CH═CH₂;
D₁ denotes a group

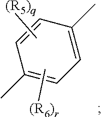

D₂ denotes a group

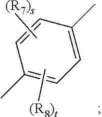

m, n, o, p, q, r, s, and t each independently denote 0, 1 or 2;
y denotes 0, 1, 2, 3, 4, 5 or 6;
z equals 0 if y equals 0 and z equals 1 if y equals 1 to 6.

In a preferred embodiment, in each of the above formulae (I), (IA), (IB), (IC) and (ID), R₁, R₂, R₃, R₄, R₅, R₆, R₇ and R₈ each independently denote C₁-C₆ alkyl (i.e., alkyl comprising 1, 2, 3, 4, 5 or 6 carbon atoms such as, e.g., methyl, ethyl, n-propyl, isopropyl, butyl, pentyl and hexyl). In an alternative embodiment, in each of the above formulae (I), (IA), (IB),(IC) and (ID) R₁, R₂, R₃, R₄, R₅, R₆, R₇ and R₈ each independently denote C₁-C₆ alkoxy (i.e., alkoxy comprising 1, 2, 3, 4, 5 or 6 carbon atoms such as, e.g., methoxy, ethoxy, n-propoxy, isopropoxy, butoxy, pentoxy and hexoxy).

In a further preferred embodiment, in each of the above formulae (I), (IA), (IB), (IC) and (ID), A₁ and A₂ each independently denote —[(CH₂)y-O]z-C(O)—CH═CH₂; R₁, R₂, R₃ and R₄ each independently denote C₁-C₆ alkyl; and m, n, o, and p each independently denote 0, 1 or 2. In an alternative embodiment, in each of the above formulae (I), (IA), (IB), (IC) and (ID), A₁ and A₂ each independently denote —[(CH₂)y-O]z-C(O)—CH═CH₂; R₁, R₂, R₃ and R₄ denote each independently C₁-C₆ alkoxy; and m, n, o, and p each independently denote 0, 1 or 2.

In another preferred embodiment, in each of the above formulae (I), (IA), (IB), (IC) and (ID), A₁ and A₂ each independently denote —C(O)-D₁-O—[(CH₂)y-O]z-C(O)—CH═CH₂ and/or —C(O)-D₂-O—[(CH₂)y-O]z-C(O)—CH═CH₂; and R₁, R₂, R₃, R₄, R₅, R₆, R₇ and R₈ each independently denote C₁-C₆ alkyl. In an alternative embodiment, in each of the above formulae (I), (IA), (IB), (IC) and (ID), A₁ and A₂ each independently denote —C(O)-D₁-O—[(CH₂)y-O]z-C(O)—CH═CH₂ and/or —C(O)-D₂-O—[(CH₂)y-O]z-C(O)—CH═CH₂; and R₁, R₂, R₃, R₄, R₅, R₆, R₇ and R₈ each independently denote C₁-C₆ alkoxy.

In another preferred embodiment, in each of the above formulae (I), (IA), (IB), (IC) and (ID), the alkyl or alkoxy group of R₁, R₂, R₃, R₄, R₅, R₆, R₇ and R₈ may comprise 1, 2, 3, 4, 5 or 6 carbon atoms.

Examples of alkyl groups comprising 4 carbon atoms include n-butyl and isobutyl. Examples of alkyl groups comprising 6 carbon atoms include hexyl, 2-methylpentyl, 3-methylpentyl, 2,2-dimethylbutyl and 2,3-dimethylbutyl.

Examples of alkoxy groups comprising 4 carbon atoms include but-1-oxy, but-2-oxy, isobutoxy and tert-butoxy. Examples of alkoxy groups comprising 6 carbon atoms comprise hex-1-oxy, hex-2-oxy, hexan-3-oxy, 2-methylpentan-1-oxy, 2-methylpentan-2-oxy, 2-methylpent-1-oxy, 2-methylpent-3-oxy, 2-methylpent-4-oxy, 4-methylpent-1-oxy, 3-methylpent-1-oxy, 3-methylpent-2-oxy, 3-methylpent-3-oxy, 3-methylpent-1-oxy, 2,2-dimethylbut-1-oxy, 2,2-dimethylbut-3-oxy, 2,2-dimethylbut-4-oxy, 4,4-dimethylbut-1-oxy, 2,3-dimethylbut-1-oxy, 2,3-dimethylbut-2-oxy, 2,3-dimethylbut-3-oxy, 2,3-dimethylbut-4-oxy, and 3,4-dimethylbut-1-oxy.

The present invention also provides a chiral liquid crystal precursor composition which comprises at least one chiral dopant of general formula (I) set forth above (including chiral dopants of any of general formulae (IA), (IB), (IC) and (ID) set forth above).

The marking of the present invention is made of a chiral liquid crystal precursor composition as set forth above and is obtainable by independently applying the chiral liquid crystal precursor composition to a substrate by a variable information printing process or by conventional printing process, applying heat to both evaporate the solvent contained in the chiral liquid crystal precursor composition and promote the chiral liquid crystal state, and hardening the applied composition in the ordered liquid crystal state. In one exemplary embodiment, the chiral liquid crystal precursor composition is applied to the surface of a substrate. Heat is then applied to both evaporate the solvent and promote the liquid crystal state. The composition in the liquid crystal state is then polymerized (cured) by irradiation with UV light or with electron beam radiation, as known by those skilled in the art. In an alternative exemplary embodiment, the heat is applied to the entire body of substrate and liquid crystal precursor composition or only to the substrate, if the latter is able to transmit the applied heat to the liquid crystal precursor composition. In an alternative exemplary embodiment, multiple different temperatures may be applied during the printing process of the chiral liquid crystal precursor composition. In a further alternative embodiment, the chiral liquid crystal precursor composition may be subjected to heat before applying it to the substrate. In an additional alternative embodiment, the step of heating the liquid crystal precursor composition and applying it to a substrate may be performed in a single step.

The chiral liquid crystal material applied in this embodiment is thus a monomeric or oligomeric precursor composition of a liquid crystal polymer. Said precursor composition comprises at least one nematic liquid crystal monomer or oligomer, said monomer or oligomer having polymerizable groups. Non-limiting examples of suitable nematic liquid crystal monomers or oligomers include bisacrylates such as
2-methyl-1,4-phenylene bis(4-(4-(acryloyloxy)butoxy)benzoate;
1,4-phenylene bis(4-(4-(acryloyloxy)butoxy)benzoate);
2-methyl-1,4-phenylene bis(4-(6-(acryloyloxy)hexyloxy)benzoate);
1,4-phenylene bis(4-((4-(acryloyloxy)butoxy)carbonyloxy)benzoate);
2-methyl-1,4-phenylene bis(4-((4-(acryloyloxy)butoxy)carbonyloxy)benzoate);
   and combinations thereof.

The nematic liquid crystal monomer or oligomer will usually be present in the precursor composition in a concentration of from 10% to 100% by weight, based on the total weight of the precursor composition.

Suitable stabilizers include Florstab UV-1 supplied by Kromachem, and Genorad 16 supplied by Rahn.

The photoinitiator will usually be present in the precursor composition in a concentration of from 0.5% to 5% by weight, based on the total weight of the precursor composition.

For obtaining cholesteric (i.e. twisted nematic) phases, said precursor composition further comprises one or more chiral dopant compounds (chiral inducers). According to the present invention, these chiral dopant compounds comprise at least one chiral dopant according to the present invention of general formula (I) set forth above (including chiral dopants of general formulae (IA), (IB), (IC) and (ID) set forth above).

Non-limiting examples of chiral dopants of general formula (I) according to the present invention include:
(3R,3aS,6S,6aS)-hexahydrofuro[3,2-b]furan-3,6-diyl bis(4-(4-(acryloyloxy)benzoyloxy)benzoate);
(3R,3aS,6S,6aS)-hexahydrofuro[3,2-b]furan-3,6-diyl bis(4-(4-(acryloyloxy)butoxy)benzoate);
(3R,3aS,6S,6aS)-hexahydrofuro[3,2-b]furan-3,6-diyl bis(4-(acryloyloxy)-2-methylbenzoate);
(3R,3aS,6S,6aS)-hexahydrofuro[3,2-b]furan-3,6-diyl bis(4-(4-(acryloyloxy)benzoyloxy)-3-methoxybenzoate);
(3R,3aS,6S,6aS)-hexahydrofuro[3,2-b]furan-3,6-diyl bis(4-(4-(acryloyloxy)-3-methoxybenzoyloxy)benzoate);
(3R,3aS,6S,6aS)-6-(4-(4-(acryloyloxy)-3-methoxybenzoyloxy)-3-methoxybenzoyloxy)hexahydrofuro[3,2-b]furan-3-yl 4-(4-(acryloyloxy)benzoyloxy)-3-methoxybenzoate;
(3R,3aS,6S,6aS)-hexahydrofuro[3,2-b]furan-3,6-diyl bis(4-(4-(acryloyloxy)-3-methoxybenzoyloxy)-3-methoxybenzoate);
(3R,3aS,6S,6aS)-hexahydrofuro[3,2-b]furan-3,6-diyl bis(4-(4-(acryloyloxy)benzoyloxy)-3-methoxybenzoate);
(3R,3aS,6S,6aS)-hexahydrofuro[3,2-b]furan-3,6-diyl bis(4-((4-(acryloyloxy)benzoyl)oxy)-3-methylbenzoate);
(3R,3aS,6S,6aS)-hexahydrofuro[3,2-b]furan-3,6-diyl bis(4-((4-(acryloyloxy)benzoyl)oxy)-3-methoxybenzoate);
(3R,3aS,6R,6aS)-hexahydrofuro[3,2-b]furan-3,6-diyl bis(4-(4-(acryloyloxy)benzoyloxy)benzoate);
(3R,3aS,6R,6aS)-hexahydrofuro[3,2-b]furan-3,6-diyl bis(4-(4-(acryloyloxy)butoxy)benzoate);
(3R,3aS,6R,6aS)-hexahydrofuro[3,2-b]furan-3,6-diyl bis(4-(acryloyloxy)-2-methylbenzoate);
(3R,3aS,6R,6aS)-hexahydrofuro[3,2-b]furan-3,6-diyl bis(4-(4-(acryloyloxy)benzoyloxy)-3-methoxybenzoate);
(3R,3aS,6R,6aS)-hexahydrofuro[3,2-b]furan-3,6-diyl bis(4-(4-(acryloyloxy)-3-methoxybenzoyloxy)benzoate);
(3R,3aS,6R,6aS)-6-(4-(4-(acryloyloxy)-3-methoxybenzoyloxy)-3-methoxybenzoyloxy)hexahydrofuro[3,2-b]furan-3-yl 4-(4-(acryloyloxy)benzoyloxy)-3-methoxybenzoate;
(3R,3aS,6R,6aS)-hexahydrofuro[3,2-b]furan-3,6-diyl bis(4-(4-(acryloyloxy)-3-methoxybenzoyloxy)-3-methoxybenzoate);
(3R,3aS,6R,6aS)-hexahydrofuro[3,2-b]furan-3,6-diyl bis(4-(4-(acryloyloxy)benzoyloxy)-3-methoxybenzoate);
(3R,3aS,6R,6aS)-hexahydrofuro[3,2-b]furan-3,6-diyl bis(4-(acryloyloxy)benzoate).

The chiral dopant compound(s) will usually be present in the precursor composition in a concentration of from 0.1% to 25% by weight, based on the total weight of the precursor composition.

A variable information printing process may be used to apply the liquid crystal precursor composition to the substrate. The term “variable information printing” encompasses variable data printing. This form of printing process is a printing process in which elements such as text, graphics or images may be changed from one printed piece to the next, allowing the “mass-customization” of items as opposed to the “mass-production” of a single item, e.g. using offset lithography, without delaying or stopping the press being necessary. A basic design is developed which comprises various sections which may be altered using a database of information that fills in the changeable fields according to the demand and intention of the user. Depending on the number of changeable fields, the final product is more or less sophisticated, e.g. like a marking according to the present invention. The elements and/or sections to be changed on each individual item can be determined in advance and controlled in each moment.

By variable information printing process it is also understood that when the marking is printed on the item or on the goods the corresponding printing will never be exactly the same even if made with the same indicia representation. For example, when printing a data matrix which is the sum of small square cells having and supporting by their arrangement an information and using the chiral liquid crystal polymer composition according to the present invention, despite the fact that the same data matrix is printed, when looking with magnification at the data matrix one will find that the cells constituting the data matrix do not have exactly the same form. While not wishing to be bound by any theory, differences in form may at least in part be attributable to the solvent evaporation from the chiral liquid crystal precursor composition which is not the same from one droplet to the next. The same applies to data matrices printed on items along a supply chain. Thus, this difference in form can further constitute a characteristic fingerprint for each data matrix. Because no two printed data matrices will ever be exactly the same, the fingerprint structure of the data matrix can be stored in a database or a repository or transformed in the form of a code for further comparison.

The precursor composition is preferably applied by ink-jet printing, either of the continuous ink-jet or of the drop-on-demand ink-jet type, preferably by single nozzle/raster. For application by ink-jet printing, the composition further contains a solvent, in order to adjust its viscosity to the low values required by these printing processes. Typical viscosity values for ink-jet printing inks are in the range of from 4 to 30 mPa·s at 25° C. Solvents which can be used include low-viscosity, slightly polar and aprotic organic solvents, such as methyl-ethyl-ketone (MEK), acetone, ethyl acetate, ethyl 3-ethoxypropionate. Chlorinated solvents like dichloromethane, trichloromethane or trichloroethylene are technically suitable, but not desirable in printing inks because of their toxicity.

The solvent will usually be comprised in the ink-jet precursor material in a range of from 10% to 95%, typically from 45% to 85% by weight.

In the case of continuous ink-jet printing, the precursor composition also comprises a dissolved conducting agent, typically a salt, such as lithium nitrate, lithium perchlorate, tetrabutylammonium chloride or tetrabutylammonium tetrafluoroborate.

The salt will usually be present in a concentration range of from 0.1% to 5% by weight.

The precursor composition preferably may further comprise security materials, which are present in low to moderate concentration, in order to increase the resistance of the marking to counterfeiting. These security materials may be selected, for example, from inorganic luminescent compounds, organic luminescent compounds, IR-absorbers, magnetic materials, forensic markers, and combinations thereof. Common concentration ranges are from 0.01% to 5% by weight for luminescent compounds, from 0.1% to 10% by weight for IR-absorbers or magnetic materials, and from 0.001% to 1% by weight for forensic marker materials.

The security materials may have a λmax of absorption or emission which is a multiple of the λmax (maximum reflection band) of the chiral liquid crystal polymer obtained from the chiral liquid crystal precursor composition.

The preferred chiral (cholesteric) liquid crystal precursor composition for carrying out the present invention using ink-jet printing equipment comprises a mixture of at least one nematic compound, at least one chiral dopant according to general formula (I) (e.g., according to formulae (IA) and/or formula (IB) and/or formula I(C) and/or formula (ID) set forth above), an organic solvent, and a photoinitiator.

The at least one nematic compound is preferably of the acrylic- or bis-acrylic type as disclosed in EP-A-0 216 712 and EP-B-0 847 432, U.S. Pat. No. 6,589,445. The preferred amount of the nematic compound present in the chiral liquid crystal precursor composition is from 10 wt % to 60 wt %, more preferably from 10 wt % to 45 wt %.

The total concentration of the one or more chiral dopants present in the chiral liquid crystal precursor composition usually ranges from 0.1 wt % to 25 wt %, preferably from 0.5 wt % to 15 wt %.

The chiral liquid crystal precursor composition for producing a marking according to the present invention may further comprise dyes, pigments, coloring agents, diluents, conducting salts, surface-active compounds, surface adhesion promoters, wetting agents, defoamers, and dispersing agents.

The marking of the present invention is preferably applied in the form of indicia representing a unique 1-dimensional, a stacked 1-dimensional, or a 2-dimensional barcode or matrix code or in the form of a glyph with specific orientation which may serve as a basis for a binary code or can support an optionally encrypted information. It may also be printed in the form of a cloud of dots where the dots are disposed in such manner that they constitute a binary code or can support optionally encrypted information. The symbology of 1-dimensional, stacked 1-dimensional or 2-dimensional barcodes or matrix codes is preferably chosen among those used in the retail industry for the marking of commercial goods. These symbologies of 1-dimensional, stacked 1-dimensional or 2-dimensional barcodes or matrix codes are internationally recognized standards, and the corresponding reading and decoding algorithms are known and implemented in commercially available devices.

Suitable 1-dimensional and stacked 1-dimensional barcode symbologies are known to the skilled person and available under symbology names such as Plessey, U.P.C., Codabar, Code 25—Non-interleaved 2 of 5, Code 25—Interleaved 2 of 5, Code 39, Code 93, Code 128, Code 128A, Code 128B, Code 128C, Code 11, CPC Binary, DUN 14, EAN 2, EAN 5, EAN 8, EAN 13, GS1-128 (formerly known as UCC/EAN-128), EAN 128, UCC 128, GS1 DataBar formerly Reduced Space Symbology (RSS), ITF-14, Pharmacode, PLANET, POSTNET, OneCode, MSI, PostBar, RM4SCC/KIX, or Telepen.

Suitable 2-dimensional barcode symbologies are known to the skilled person and available under symbology names such as 3-DI, ArrayTag, Aztec Code, Small Aztec Code, bCODE, Bullseye, Codablock, Code 1, Code, 16K, Code 49, Color code, CP Code, DataGlyphs, Datamatrix, Datastrip Code, Dot Code A, EZcode, High Capacity Color Barcode, HueCode, INTACTA.CODE, InterCode, MaxiCode, mCode, MiniCode, PDF417, Micro PDF417, PDMark, PaperDisk, Optar, QR Code, Semacode, SmartCode, Snowflake Code, ShotCode, SuperCode, Trillcode, UltraCode, VeriCode, VSCode, WaterCode, and ECC200. This latter has a built-in error correction code and is defined in international standard ISO/CEI 16022:2006.

Suitable font types for optical character recognition (OCR) are known to the skilled person.

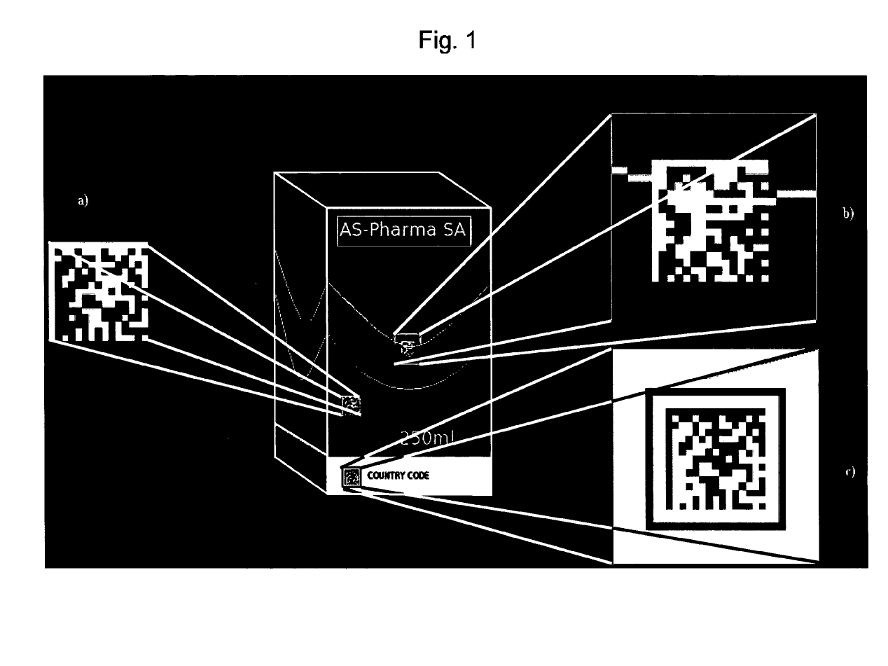

FIG. 1 schematically shows a product packaging having a chiral liquid crystal marking of the present invention thereon. The marking is present in the form of a ECC200 data matrix code on the surface of said packaging. Data Matrix ECC200 is a public domain symbology. The marking can be applied to any desired position on the packaging. Thus it can be present wholly on a first background color (a), or partially, overlapping with a first background color and a second design color motive present on the packaging (b), or wholly be present on a white or colorless region of the packaging (c).

The reading device for reading the marking of the present invention can be constructed on the basis of commercially available barcode readers, in particular on the basis of hand-held CCD/CMOS-camera reading equipment and reading stations used in the retail industry. In case of an appropriate matching of the marking with the available (narrow-band) illumination, said reading equipment may be directly enabled to read the liquid crystal codes.

In other cases, the reading device can be further adapted (enabled) so as to read the response of the specific security elements implemented in the marking. Correspondingly adapted flat-bed scanners can also be used. CCD-camera based barcode readers are known to the skilled person and are produced by several industrial companies, such as AccuSort, Cognex, DVT, Microscan, Omron, Sick, RVSI, Keyence, etc.

Said adaptation of the reading device may comprise the implementation of one or several optical filters chosen from linear polarizing filters, right-circular polarizing filters, left-circular polarizing filters, electro-optic polarization filters, wave-plates, and spectrally selective color filters of any type, and combinations thereof. In a particular embodiment at least two different optical filters are used. Said adaptation may further comprise the implementation of one or several particular light sources chosen from spectrally selective (i.e. colored) light sources, linear polarized light sources, left- and right-circular polarized light sources, and combinations thereof.

The light sources may be chosen from ambient light, incandescent light, laser diodes, light emitting diodes, and all types of light sources having color filters. Said light sources may have an emission spectrum in the spectral domain of the visible light (400-700 nm wavelength), the near optical infrared (700-1100 nm wavelength), the far optical infrared (1100-2500 nm wavelength), or the UV (200-400 nm wavelength) region of the electromagnetic spectrum.

Said reading device is thus not only enabled to read the marking, but also to authenticate it as being made of the correct security material, i.e. comprising the required security elements. Said reading device delivers a digital information representative of the code which has been read, and pointing towards an entry in a database corresponding to the item carrying said marking and code.

Said digital information may be compared with information stored in the reading device or may be exchanged between the reading device and an external database; the exchange can take place in encrypted form, using e.g. a public/private encoding of the RSA type. Said exchange of information can take place by all kinds of transmission means, e.g. wire-bound transmission, wireless radio link, infrared-link, etc.

Said coating composition may preferably comprise further security materials, which are present in low to moderate concentration, in order to increase its resistance to counterfeiting. These security materials may be selected from, for example, inorganic luminescent compounds, organic luminescent compounds, IR-absorbers, magnetic materials, forensic markers, and combinations thereof. Typical concentration ranges are from 0.01% to 5% by weight for luminescent compounds, from 0.1% to 10% by weight for IR-absorbers or magnetic materials, and from 0.001% to 1% by weight for forensic marker materials. The security materials may, for example, have a λmax of absorption or emission which is a multiple of the λmax (maximum reflection band) of the chiral liquid crystal polymer obtained from the chiral liquid crystal precursor composition.

To accommodate particular conditions, the precursor composition for producing a marking according to the present invention may further comprise dyes, pigments, coloring agents, diluents, conducting salts, surface-active compounds, surface adhesion promoters, wetting agents, defoamers, and dispersing agents, as known in the art.

The authentication and identification of the chiral liquid crystal marking according to the present invention requires a light source and may be done in one of the following ways:

i) by illuminating the marking with circular or linear polarized light and detecting the marking's reflection;

ii) by illuminating the marking with non-polarized (e.g. ambient) light and detecting the marking's reflection through a circular or linear polarizing filter,

iii) by a combination of circular or linear polarized illumination and detection through a circular or linear polarizing filter.

The illumination of a marked item or article is thus performed by a light source chosen from non-polarized light sources, linear polarized light sources, left circular polarized light sources, and right circular polarized light sources.

In all cases, detection may be performed by the human eye or with the help of electro-optic detection equipment, such as a photocell, or a CCD or CMOS camera. The light sources and the detection may be made or chosen spectrally selective through the use of particular light emitters and/or color filters. The detection is preferably carried out in the visible region (400-700 nm wavelength) of the electromagnetic spectrum.

In a particular embodiment, the illumination of the marking for authenticating an item or article is performed using at least two different light sources selected from non-polarized (randomly polarized) light sources, linear polarized light sources, left circular polarized light sources, and right circular polarized light sources.

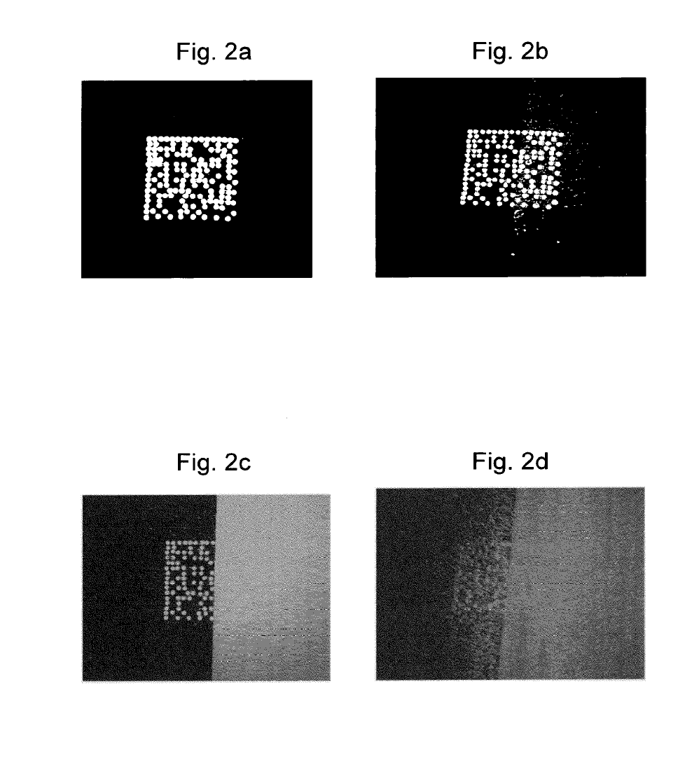

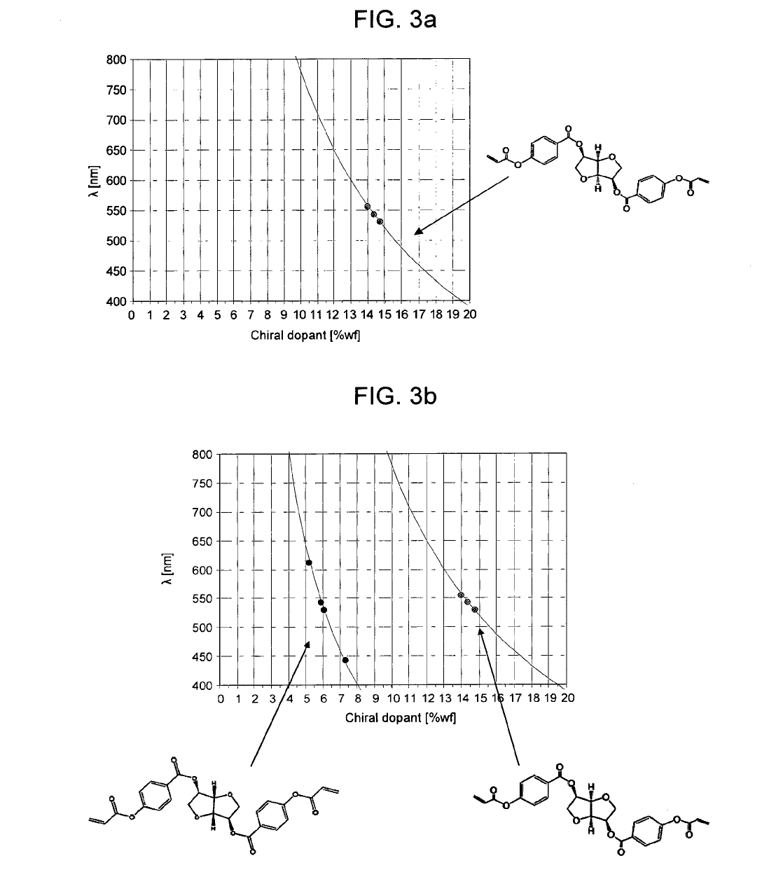

FIG. 2 shows images taken from ECC200 Data matrix codes printed with liquid crystal material on coated cardboard. These images clearly illustrate the advantage of using the polarizing properties of the chiral liquid crystal material marking for the reading of the printed code on a clear or structured background. Most advantageous is a combination of polarized light for illumination and the use of a polarizing filter in front of the camera. All images were taken with the same light source and with the same camera settings, all in black and white mode, and with or without polarizing filters before the light source and/or the camera. The images were digitally treated for maximum contrast and optimum brightness.

In a preferred embodiment, the chiral liquid crystal marking of the present invention is rendered visible under non-polarized (preferably ambient) light by a passive detecting means such as a linear or a circular polarizing filter. The marking can, however, also be identified and authenticated outside the visible spectrum (400-700 nm wavelength), e.g. in the infrared region (700 to 2500 nm wavelength), preferably in the near optical infrared (700-1100 nm wavelength), in the far optical infrared (1100-2500 nm wavelength) or in the UV (200-400 nm wavelength) region of the electromagnetic spectrum, provided that the marking has a reflection band in these regions.

The chiral (cholesteric) liquid crystal polymer is, by its nature, a spectrally selective reflector whose reflection band can be tuned across part of the electromagnetic spectrum by an appropriate choice of its helical pitch. Said pitch noteworthy depends on the ratio of nematic precursor material to chiral dopant in the liquid crystal precursor, and on the temperature of polymerization. After polymerization, the helical pitch, and hence the reflection color of the material, remains fixed.

As known to the skilled person, low amounts of chiral dopant result in a low helical twisting and hence in a large helical pitch. Therefore, low amounts of chiral dopant generate a reflection band of the resulting polymer at the long wavelength end of the spectrum, typically in the infrared or red region, whereas higher amounts of chiral dopant generate a reflection band of the resulting polymer at the short wavelength end of the spectrum, typically in the blue or UV region.

Attention must also be paid to the handedness of the chiral dopant, i.e. whether a selected dopant results in a left or in a right helical pitch, resulting in respective opposite circular polarization of the reflected light. For example, isomannide derivatives are known to induce the reflection of left circular polarized light, whereas isosorbide derivatives are known to induce the reflection of right circular polarized light.

Example of the Preparation of a Chiral Dopant According to General Formula (I):

### Preparation of (3R,3aS,6S,6aS)-hexahydrofuro[3,2-b]furan-3,6-diyl bis(4-(acryloyloxy)benzoate)

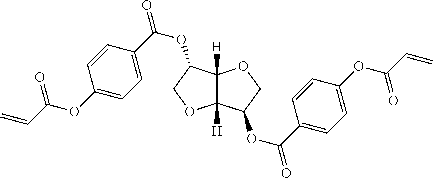 [SMILES: C=CC(=O)Oc1ccc(C(=O)O[C@H]2CO[C@@H]3[C@H]2OC[C@H]3OC(=O)c2ccc(OC(=O)C=C)cc2)cc1]

The benzoate derivative is prepared as follows:

Step 1:

In a 2 L round-bottomed flask, 4-hydroxybenzoic acid (35 g) is added to vigorously stirred water (280 mL) under nitrogen, followed by slow addition of potassium hydroxide (18.5 g). At 15° C., potassium carbonate (35 g) and isopropanol (90 mL) are added and the reaction mixture is then cooled down to −15° C. 3-Chloropropionyl chloride (27.8 mL) is incorporated into the reaction mixture, keeping the temperature at values lower than −10° C. The reaction mixture is allowed to stir for 20 minutes, followed by the addition of 2-butanone (250 mL) and BHDMA (70 mg), and finally is poured into a 6N solution of HCl (100 mL). As a result, the formation of a white precipitate is observed. Addition of 10% NaCl aqueous solution (90 mL) allows better separation between the two phases present. The isolated organic phase is treated to remove 2-butanone and isopropanol under vacuum. A yellowish solid is obtained which is washed first with toluene (350 mL) and later on with n-heptane (90 mL) leading to the formation of 4-((3-chloropropanoyl)oxy)benzoic acid in 84% yield (48 g).

Step 2:

In a 1 L round-bottomed flask, 4-((3-chloropropanoyl)oxy)benzoic acid (45 g) and BHT (0.13 g) are added to toluene (230 mL). The solution is gradually warmed at 70° C. SOCl₂ (17.1 mL) is slowly added, keeping control on the temperature. The reaction mixture is then allowed to stir at 80° C. for 3 hours, followed by the removal of the toluene and excess SOCl₂, which leads to the formation of the desired product in 95% yield (46 g)

Step 3:

In a 25 mL round-bottomed flask, 1,4:3,6-dianhydro-L-glucitol (6.8 mmol), triethylamine (4.6 mmol) in toluene (10 mL) are combined. After a few minutes of stirring, 4-(chlorocarbonyl)phenyl acrylate (14.4 mmol) is added and the reaction mixture is allowed to stir at 80° C. overnight. Water (10 mL) is added to the mixture, and then the organic solvent is removed under vacuum. The crude product is purified by flash chromatography leading to the desired compound in 52% yield (1.75 g). Mass spectroscopy analysis provide the correct mass of the product: M+1=495

The process described above can also be represented as follows:

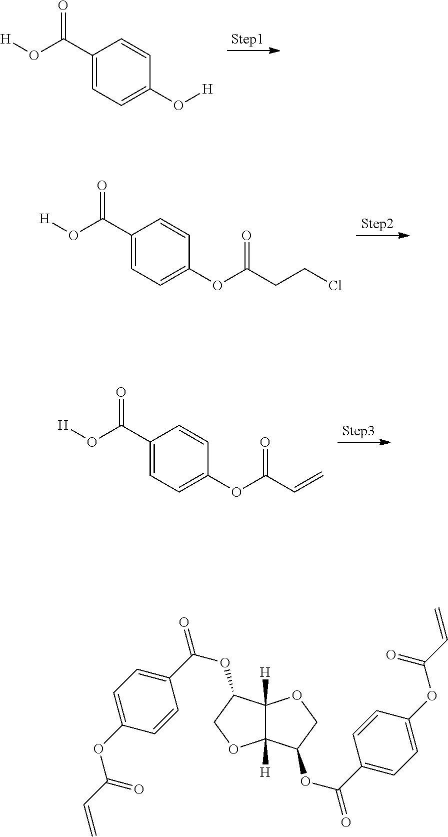 [SMILES: O=C(O)c1ccc(O)cc1 ; O=C(CCCl)Oc1ccc(C(=O)O)cc1 ; C=CC(=O)Oc1ccc(C(=O)O)cc1 ; C=CC(=O)Oc1ccc(C(=O)O[C@H]2CO[C@@H]3[C@H]2OC[C@H]3OC(=O)c2ccc(OC(=O)C=C)cc2)cc1]

The following is a typical example of a chiral liquid crystal polymer precursor composition that can be applied by the continuous ink-jet printing process:

### EXAMPLE

| Composition | Weight % |
|---|---|
| Acetone | 52.25 |
| 2-methyl-1,4-phenylene bis(4-(4- | 42.8 |
| (acryloyloxy)butoxy)benzoate) | |
| (3R,3aS,6S,6aS)-hexahydrofuro[3,2-b]furan-3,6-diyl bis(4- | 3.5 |
| (acryloyloxy)benzoate) | |
| Irgacure 907 | 1.0 |
| LiClO4 | 0.45 |

The following is a typical example of a cholesteric liquid crystal polymer precursor composition, which can be applied by a flexographic printing process.

A chiral liquid crystal precursor composition (I) was prepared as follows, the indicated percentages being by weight based on the total weight of the composition:

Chiral dopant compound (3R,3aR,6R,6aR)-hexahydrofuro[3,2-b]furan-3,6-diyl bis(4-(acryloyloxy)benzoate)shown below (7.11%), nematic compound 2-methyl-1,4-phenylene bis(4-(4-(acryloyloxy)butoxy)benzoate) (41.04%)), and cyclopentanone (50.55%) were placed in a flask which was thereafter heated at 40° C. until a solution was obtained. To the solution were added 2-methyl-1[4-(methylthio)phenyl]-2-morpholinopropan-1-one (Irgacure 907® from Ciba, photoinitiator, 1.08%) and isopropylthioxanthone (photoinitiator, 0.22%). The final mixture was stirred until complete dissolution was achieved to result in the chiral liquid crystal precursor composition (I).

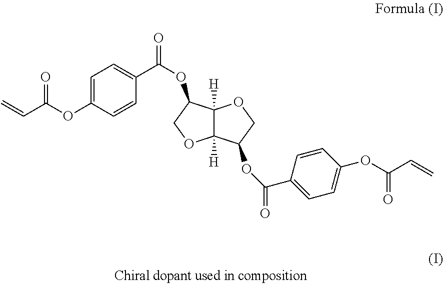 [SMILES: C=CC(=O)Oc1ccc(C(=O)O[C@@H]2CO[C@H]3[C@@H]2OC[C@H]3OC(=O)c2ccc(OC(=O)C=C)cc2)cc1]

Preparation of a layer of a cured chiral liquid crystal precursor composition:

The above composition (I) was coated on a substrate (transparent PET film, thickness 125 μm) and the resultant layer was heated to about 85° C. for about 30 seconds to evaporate the solvent and to develop a chiral liquid crystal phase, i.e., a state that shows a specific reflection band whose position depends on the concentration of the chiral dopant (3R,3aR,6R,6aR)-hexahydrofuro[3,2-b]furan-3,6-diyl bis(4-(acryloyloxy)benzoate) in the composition. Thereafter the composition was cured by irradiation with a UV lamp (mercury low-pressure lamp having a UV irradiance of 10 mW/cm²) for about 1 second to freeze the cholesteric liquid crystal phase through co-polymerization of the polymerizable groups of nematic and chiral dopant compounds. After the curing the composition was substantially free of solvent (only trace amounts of cyclopentanone were present)

The concentration of chiral dopant in the chiral liquid crystal precursor composition allows control of the position of the selective reflection band and as a result thereof, the color of the cured chiral liquid crystal layer. This is illustrated by FIG. 3a which is a diagram showing the wavelength of the maximum normal reflection as a function of the concentration of the chiral dopant in the dry composition. As can be seen from FIG. 3a, with 14.38% of chiral dopant compound the wavelength of the maximum normal reflection of the composition is around 542 nm, which affords a green color of the corresponding layer. As shown in FIG. 3a, increasing (decreasing) the concentration of chiral dopant in the composition results in a decrease (increase) of the wavelength of the maximum normal reflection.

A chiral liquid crystal precursor composition (II) was prepared as follows, the indicated percentages being by weight based on the total weight of the composition:

Chiral dopant (3R,3aS,6S,6aS)-hexahydrofuro[3,2-b]furan-3,6-diyl bis(4-(acryloyloxy)benzoate) shown below (2.96%), nematic compound 2-methyl-1,4-phenylene bis(4-(4-(acryloyloxy)butoxy)benzoate) (45.66%), and cyclopentanone (50.08%) were placed in a flask which was thereafter heated at 40° C. until a solution was obtained. To the solution were added 2-methyl-1[4-(methylthio)phenyl]-2-morpholinopropan-1-one (Irgacure 907® from Ciba, photoinitiator, 1.08%) and isopropylthioxanthone (photoinitiator, 0.22%). The final mixture was stirred until complete dissolution was achieved to result in the chiral liquid crystal precursor composition (II).

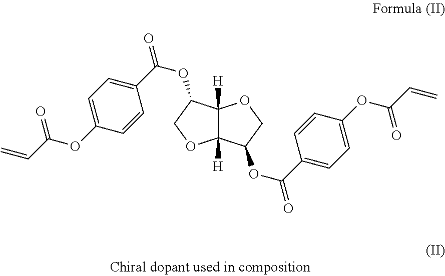 [SMILES: C=CC(=O)Oc1ccc(C(=O)O[C@H]2CO[C@@H]3[C@H]2OC[C@H]3OC(=O)c2ccc(OC(=O)C=C)cc2)cc1]

Preparation of a layer of a cured chiral liquid crystal precursor composition:

The above composition (II) was coated on a substrate (transparent PET film, thickness 125 μm) and the resultant layer was heated to about 85° C. for about 30 seconds to evaporate the solvent and to develop a cholesteric liquid crystal phase, i.e., a state that shows a specific reflection band whose position depends on the concentration of the chiral dopant compound in the composition. Thereafter the composition was cured by irradiation with a UV lamp (mercury low-pressure lamp having a UV irradiance of 10 mW/cm2) for about 1 second to freeze the cholesteric liquid crystal phase through co-polymerization of the polymerizable groups of chiral dopant and nematic compounds. After the curing the composition was substantially free of solvent (only trace amounts of cyclopentanone were present).

The concentration of chiral dopant compound in the chiral liquid crystal precursor composition allows control of the position of the selective reflection band and as a result thereof, the color of the cured chiral liquid crystal precursor layer. This is illustrated by FIG. 3b which is a diagram showing the wavelength of the maximum normal reflection as a function of the concentration of the chiral dopant compound in the dry composition. As can be seen from FIG. 3b, with 5.93% of chiral dopant compound the wavelength of the maximum normal reflection of the composition is around 543 nm, which affords a green color of the corresponding layer. As shown in FIG. 3b, increasing (decreasing) the concentration of chiral dopant compound in the composition results in a decrease (increase) of the wavelength of the maximum normal reflection.

As can further be taken from FIG. 3b, in order to obtain the same green color, only 5.93% of chiral dopant used in composition (II) is required while for chiral dopant used in composition (I) a concentration of 14.38% is necessary.

The advantage provided by the chiral dopant of formula (I) set forth above in comparison of a chiral dopant of the prior art thus is self-explanatory. It was observed that compared to the chiral dopant of the prior art, the helical twisting power (HTP) of the chiral dopant according to the present invention is about twice or about third times higher, the time for obtaining the chiral liquid crystal polymer film is reduced and the need to obtain the same quality requires less concentration of chiral dopant of formula (I).

The examples presented above are merely intended to illustrate the present invention and should in no way be considered to limit the scope of the claims appended hereto.

1. Chiral dopant of general formula (I): 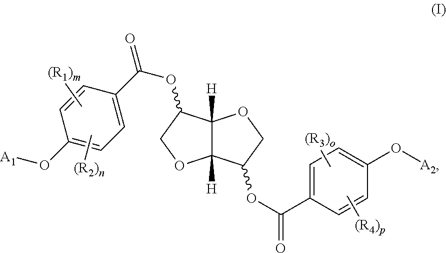
wherein
R₁, R₂, R₃, R₄, R₅, R₆, R₇ and R₈ each independently denote C₁-C₆ alkyl or C₁-C₆ alkoxy;
A₁ and A₂ each independently denote a group:
(i) —[(CH₂)y-O]z-C(O)—CH═CH₂;
(ii)—C(O)-D₁-O—[(CH₂)y-O]z-C(O)—CH═CH₂;
(iii) —C(O)-D₂-O—[(CH₂)y-O]z-C(O)—CH═CH₂;
D₁ denotes a group 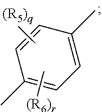
D₂ denotes a group 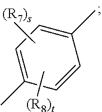
m, n, o, p, q, r, s, and t each independently denote 0, 1 or 2;
y denotes 0, 1, 2, 3, 4, 5 or 6;
z equals 0 if y equals 0 and z equals 1 if y equals 1 to 6.

2. Chiral dopant according to claim 1, having general formula (IA): 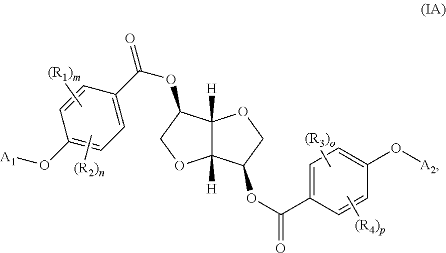
wherein
R₁, R₂, R₃, R₄, R₅, R₆, R₇ and R₈ each independently denote C₁-C₆ alkyl or C₁-C₆ alkoxy;
A₁ and A₂ each independently denote a group:
(i) —[(CH₂)y-O]z-C(O)—CH═CH₂;
(ii) —C(O)-D₁-O—[(CH₂)y-O]z-C(O)—CH═CH₂;
(iii) —C(O)-D₂-O—[(CH₂)y-O]z-C(O)—CH═CH₂;
D₁ denotes a group 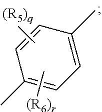
D₂ denotes a group 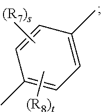
m, n, o, p, q, r, s, and t each independently denote 0, 1 or 2;
y denotes 0, 1, 2, 3, 4, 5 or 6;
z equals 0 if y equals 0 and z equals 1 if y equals 1 to 6.

3. Chiral dopant according to claim 1, having general formula (IB): 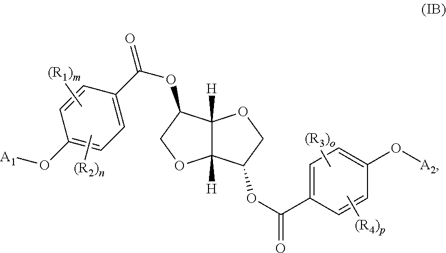
wherein
R₁, R₂, R₃, R₄, R₅, R₆, R₇ and R₈ each independently denote C₁-C₆ alkyl or C₁-C₆ alkoxy;
A₁ and A₂ each independently denote a group:
(i) —[(CH₂)y-O]z-C(O)—CH═CH₂;
(ii) —C(O)-D₁-O—[(CH₂)y-O]z-C(O)—CH═CH₂;
(i) —C(O)-D₂-O—[(CH₂)y-O]z-C(O)—CH═CH₂;
D₁ denotes a group 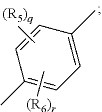
D₂ denotes a group 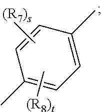
m, n, o, p, q, r, s, and t each independently denote 0, 1 or 2;
y denotes 0, 1, 2, 3, 4, 5 or 6;
z equals 0 if y equals 0 and z equals 1 if y equals 1 to 6.

4. Chiral dopant according to claim 1, having general formula (IC): 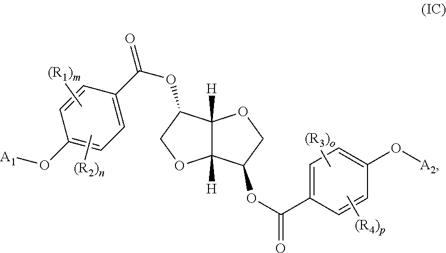
wherein
R₁, R₂, R₃, R₄, R₅, R₆, R₇ and R₈ each independently denote C₁-C₆ alkyl or C₁-C₆ alkoxy;
A₁ and A₂ each independently denote a group:
(i) —[(CH₂)y-O]z-C(O)—CH═CH₂;
(ii) —C(O)-D₁-O—[(CH₂)y-O]z-C(O)—CH═CH₂;
(iii) —C(O)-D₂-O—[(CH₂)y-O]z-C(O)—CH═CH₂;
D₁ denotes a group 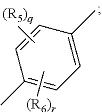
D₂ denotes a group 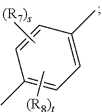
m, n, o, p, q, r, s, and t denote each independently 0, 1 or 2;
y denotes 0, 1, 2, 3, 4, 5 or 6;
z equals 0 if y equals 0 and z equals 1 if y equals 1 to 6.

5. Chiral dopant according to claim 1, having general formula (ID): 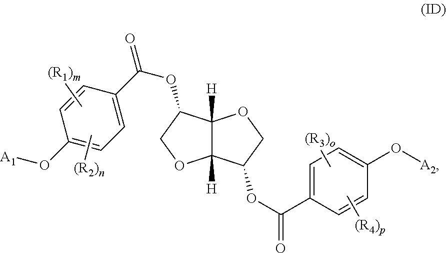
wherein
R₁, R₂, R₃, R₄, R₅, R₆, R₇ and R₈ each independently denote C₁-C₆ alkyl or C₁-C₆ alkoxy;
A₁ and A₂ each independently denote a group:
(i) —[(CH₂)y-O]z-C(O)—CH═CH₂;
(ii) —C(O)-D₁-O—[(CH₂)y-O]z-C(O)—CH═CH₂;
(iii) —C(O)-D₂-O—[(CH₂)y-O]z-C(O)—CH═CH₂;
D₁ denotes a group 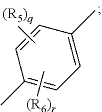
D₂ denotes a group 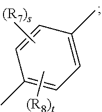
m, n, o, p, q, r, s, and t each independently denote 0, 1 or 2;
y denotes 0, 1, 2, 3, 4, 5 or 6;
z equals 0 if y equals 0 and z equals 1 if y equals 1 to 6.

6. Chiral dopant according to claim 1, wherein R₁, R₂, R₃, R₄, R₅, R₆, R₇ and R₈ each independently denote C₁-C₆ alkyl.

7. Chiral dopant according to claim 1, wherein R₁, R₂, R₃, R₄, R₅, R₆, R₇ and R₈ each independently denote C₁-C₆ alkoxy.

8. Chiral dopant according to claim 1, wherein A₁ and A₂ each independently denote —[(CH₂)y-O]z-C(O)—CH═CH₂; R₁, R₂, R₃ and R₄ each independently denote C₁-C₆ alkyl or C₁-C₆ alkoxy; and m, n, o, and p each independently denote 0, 1, or 2.

9. Chiral dopant according to claim 1, wherein A₁ and A₂ each independently denote —C(O)-D₁-O—[(CH₂)y-O]z-C(O)—CH═CH₂ and/or —C(O)-D₂-O—[(CH₂)y-O]z-C(O)—CH═CH₂; and R₁, R₂, R₃, R₄, R₅, R₆, each independently denote C₁-C₆ alkyl or C₁-C₆ alkoxy.

10. Chiral dopant according to claim 1, wherein the alkyl or alkoxy groups of R₁, R₂, R₃, R₄, R₅, R₆, R₇ and R₈ each independently comprise 1, 2, 3, 4, 5 or 6 carbon atoms.

11. Chiral liquid crystal precursor composition comprising at least one or more chiral dopants according to claim 1.

12. Chiral liquid crystal precursor composition according to claim 11, wherein the composition comprises in addition one or more nematic components.

13. Chiral liquid crystal precursor composition according to claim 11 containing a security materials selected from inorganic luminescent compounds, organic luminescent compounds, IR-absorbers, magnetic materials, forensic markers, and combinations thereof.

14. Marking for an item or article, wherein said marking comprises chiral polymeric liquid crystal material wherein the polymer of the chiral polymeric liquid crystal material comprises units derived from one or more chiral dopants according to claim 1.

15. Item or article carrying at least one marking according to claim 14.

16. Use of a marking according to claim 14 for tracking or tracing an item or article.

17. Intermediate in the liquid crystal state, obtainable by independently applying a liquid crystal precursor composition comprising a chiral dopant according to claim 1 to a substrate by a variable information printing process or a conventional printing process and applying heat to both evaporate the solvent contained in the liquid crystal precursor composition and promote the liquid crystal state.

18. An ink, coating or flake comprising one or more nematic compounds and one or more chiral dopants according to claim 1.
